(19) United States
(12) Patent Application Publication
(10) Pub. No.: US 2026/0169002 A1
(43) Pub. Date: Jun. 18, 2026

(54) PROFILING NEWLY-SYNTHESIZED GLYCOPROTEINS AS MOLECULAR SIGNATURES OF INJURY IN DONOR ORGANS

(71) Applicants: Carnegie Mellon University, Pittsburgh, PA (US); UNIVERSITY OF PITTSBURGH - OF THE COMMONWEALTH SYSTEM OF HIGHER EDUCATION, Pittsburgh, PA (US)

(72) Inventors: Xi Ren, Wexford, PA (US); Zihan Ling, Pittsburgh, PA (US); Kentaro Noda, Pittsburgh, PA (US); Pablo Sanchez, Pittsburgh, PA (US)

(21) Appl. No.: 19/126,765

(22) PCT Filed: Nov. 3, 2023
(86) PCT No.: PCT/US2023/078655

§ 371 Date: May 2, 2025

Related U.S. Application Data
(60) Provisional application No. 63/422,484, filed on Nov. 4, 2022.

(51) Int. Cl. G01N 33/68; G01N 33/50

(52) U.S. Cl. CPC G01N 33/6842; G01N 33/5088; G01N 33/6893; G01N 2400/00; G01N 2440/32; G01N 2440/38; G01N 2800/245

(57) ABSTRACT

Methods and materials for improving outcomes for ex vivo perfused organs are provided herein. For example, this document provides methods and materials that can be used to assess and treat lungs during ex vivo perfusion prior to transplantation.

### CROSS-REFERENCE TO RELATED APPLICATIONS

This application claims priority from U.S. Provisional Application Ser. No. 63/422,484, filed on Nov. 4, 2022. The disclosure of the prior application is considered part of (and is incorporated by reference in) the disclosure of this application.

### STATEMENT AS TO FEDERALLY SPONSORED RESEARCH

This invention was made with United States government support under HL158969 awarded by the National Institutes of Health. The U.S. government has certain rights in the invention.

### TECHNICAL FIELD

This document relates to methods and materials for improving outcomes for ex vivo perfused organs. For example, this document provides methods and materials that can be used to assess and treat lungs during ex vivo perfusion prior to transplantation.

### BACKGROUND

Warm ischemia injury (WII) is a critical pathogenic event in major organs, often resulting in acute severe organ dysfunction (e.g., myocardial infarction) (Yellon and Hausenloy, 2007, New Engl J Med., 357:1121-1135). In transplantation of lungs, for example, WII can render lung grafts susceptible to developing primary graft dysfunction, a leading cause of early mortality in transplant recipients (Levvey et al., 2019, J Heart Lung Transplant., 38:26-34). WII also can occur in lung grafts during anastomosis in surgery, or during an organ donation process known as donation after circulatory death (Christie et al., 2005, J Heart Lung Transpl., 24:1454-1459; Cypel et al., 2015, J Heart Lung Transplant. 34:1278-1282; Healey et al., 2020, Am J Transplant., 20:1574-1581; Levvey et al., supra; Mooney et al., 2016, Am J Transplant., 16:1207-1215; Qaqish et al., 2021, J Thor Cardiovasc Surg., 161:1546-1555.e1541; Shaver and Ware, 2017, Exp Rev Resp Med., 11:119-128; Valdivia et al., 2019, Clin Transplant., 33:e13561; and Villavicencio et al., 2018, Ann Thor Surg., 106:1619-1627). Clinically, lungs with WII are more likely to exhibit progressive functional decline during evaluation on the platform of ex vivo lung perfusion (EVLP), and subsequently become disqualified for transplantation (De Vleeschauwer et al., 2011, J Heart Lung Transplant., 30:975-981; Divithotawela et al., 2019, JAMA Surg., 154:1143-1150; Loor et al., 2019, Lancet Resp Med., 7:975-984; Verzelloni Sef et al., 2022, Clin Transplant., 36:e14468; and Whitson et al., 2018, J Heart Lung Transplant., 37:S147-S148). Other types of injury (e.g., inflammatory injury, traumatic injury, resuscitation injury, aspiration-related injury, pneumonia, viral and/or bacterial infection, hyperoxic lung injury, neurogenic pulmonary edema, (poly-)substance exposure, smoking-associated injury, and ventilator-associated lung injury) also can adversely affect the lungs and, in some cases, other major organs (e.g., heart, kidneys, and liver). The biological effectors of WII and other injuries that influence graft viability, or treatment options alleviating such graft injury, have not been clearly identified, even through the use of transcriptomic or multi-omics analyses.

### SUMMARY

This document is based, at least in part, on the development of a bioanalytical method for labeling, enriching, and analyzing glycoproteins newly synthesized in lungs over EVLP period. As demonstrated herein, the method can enable sensitive proteomic detection of subtle changes in glycoproteins within a potential donor organ (also referred to as a “graft”) during EVLP for as short as four hours, and the method was used to identify molecular mediators of WII that influence lung graft viability during EVLP. For example, as described herein using a rat lung WII model, NewS-glycoproteomes were compared between WII and control lung grafts during EVLP, and a highly specific subset of proteins with WII-associated upregulation or downregulation of their synthesis were identified. Among those candidates, inhibition of the calcineurin pathway was demonstrated to attenuate WII and improve lung transplant outcomes.

This document provides methods and materials that can be used to determine whether an organ (e.g., a lung) removed from a mammal (e.g., a human) is suitable for transplant into another mammal (e.g., another human), or if the organ is likely to have a graft injury (e.g., WII) and therefore may not be suitable for transplant without further treatment. For example, organs determined to have WII using a method described herein can be treated as described herein (e.g., with a calcineurin pathway inhibitor) to alleviate the WII, and the treated organ can then be transplanted into a second mammal. Having the ability to detect markers of graft injury (e.g., WII) with high sensitivity and in a relatively short period of time provides a unique and unrealized opportunity for rapid and accurate determination of whether an organ is suitable for transplant, and for treating an organ to improve its suitability for transplant.

In a first aspect, this document features a method for assessing an organ or a portion thereof removed from a mammal for transplant suitability. The method can include, or consist essentially of.
(a) perfusing the organ or the portion thereof with an aqueous solution containing (i) an azide-containing chemical probe, thereby azide-tagging glycosylated polypeptides synthesized within cells of the organ or the portion thereof during the perfusing, or (ii) an alkyne-containing chemical probe, thereby alkyne-tagging glycosylated polypeptides synthesized within cells of the organ or the portion thereof during the perfusing;
(b) obtaining a sample of the organ after the perfusing, and generating an extract containing the azide- or alkyne-tagged glycosylated polypeptides;
(c) contacting the azide- or alkyne-tagged glycosylated polypeptides in the extract with desthiobiotin, thereby generating desthiobiotinylated glycosylated polypeptides, wherein the desthiobiotin is alkyne-desthiobiotin when the glycosylated polypeptides are azide-tagged, and wherein the desthiobiotin is azide-desthiobiotin when the glycosylated polypeptides are alkyne-tagged;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of graft injury; and
(h) determining that the organ is suitable for transplant when the eluate does not contain altered levels of the one or more desthiobiotinylated glycoprotein markers of the graft injury, as compared to a control eluate from a corresponding organ without the graft injury, or determining that the organ may not be suitable for transplant when the eluate contains altered levels of the one or more desthiobiotinylated glycoprotein markers of the graft injury, as compared to a control eluate from a corresponding organ without the graft injury.

The organ can be a lung, the altered levels can be elevated levels of the desthiobiotinylated glycoprotein markers, the graft injury can include warm ischemia injury, and the one or more desthiobiotinylated glycoprotein markers can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12. The organ can be a lung, a kidney, a heart, or a liver or a portion thereof. The chemical probe can include one or more of N-azidoacetylgalactosamine-tetraacylated, N-azidoacetylglucosamine-tetraacylated, and N-azidoacetylmannosamine-tetraacylated, and the desthiobiotin can include alkyne-desthiobiotin. The chemical probe can include one or more of N-(4-pentynoyl)-galactosamine tetraacylated, N-(4-pentynoyl)-glucosamine tetraacylated, and N-(4-pentynoyl)-mannosamine-tetraacylated, and the desthiobiotin can include azide-desthiobiotin. The mammal can be a human. The method can include perfusing the organ for about 2 to about 6 hours, or perfusing the organ for about 4 hours.

In another aspect, this document features a method for assessing and treating an organ removed from a mammal, where the method includes, or consists essentially of:
(a) perfusing the organ or the portion thereof with an aqueous solution containing (i) an azide-containing chemical probe, thereby azide-tagging glycosylated polypeptides synthesized within cells of the organ or the portion thereof during the perfusing, or (ii) an alkyne-containing chemical probe, thereby alkyne-tagging glycosylated polypeptides synthesized within cells of the organ or the portion thereof during the perfusing;
(b) obtaining a sample of the organ after the perfusing, and generating an extract containing the azide- or alkyne-tagged glycosylated polypeptides;
(c) contacting the azide- or alkyne-tagged glycosylated polypeptides in the extract with desthiobiotin, thereby generating desthiobiotinylated glycosylated polypeptides, wherein the desthiobiotin is alkyne-desthiobiotin when the glycosylated polypeptides are azide-tagged, and wherein the desthiobiotin is azide-desthiobiotin when the glycosylated polypeptides are alkyne-tagged;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of graft injury;
(h) determining that the eluate contains altered levels of the one or more desthiobiotinylated glycoprotein markers of graft injury, as compared to a control eluate from a corresponding organ without graft injury; and
(i) treating the organ with a calcineurin pathway inhibitor.

The organ can be a lung, the altered levels can be elevated levels, the graft injury can include warm ischemia injury, and the one or more desthiobiotinylated glycoprotein markers of graft injury can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12. The organ can be a lung, a kidney, a heart, or a liver or a portion thereof. The chemical probe can include one or more of N-azidoacetylgalactosamine-tetraacylated, N-azidoacetylglucosamine-tetraacylated, and N-azidoacetylmannosamine-tetraacylated, and the desthiobiotin can include alkyne-desthiobiotin. The chemical probe can include one or more of N-(4-pentynoyl)-galactosamine tetraacylated, N-(4-pentynoyl)-glucosamine tetraacylated, and N-(4-pentynoyl)-mannosamine-tetraacylated, and the desthiobiotin can include azide-desthiobiotin. The mammal can be a human. The method can include perfusing the organ for about 2 to about 6 hours, or perfusing the organ for about 4 hours. The calcineurin pathway inhibitor can include one or more of cyclosporine A (CyA), tacrolimus, pimecrolimus, and voclosporin.

In another aspect, this document features a method for assessing an organ or a portion thereof removed from a mammal for transplant suitability, where the method includes, or consists essentially of.
(a) perfusing the organ or the portion thereof with an aqueous solution containing an azide-containing chemical probe, thereby azide-tagging glycosylated polypeptides synthesized within cells of the organ or the portion thereof during the perfusing;
(b) obtaining a sample of the organ after the perfusing, and generating an extract containing the azide-tagged glycosylated polypeptides;
(c) contacting the azide-tagged glycosylated polypeptides in the extract with alkyne-desthiobiotin, thereby generating desthiobiotinylated glycosylated polypeptides;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of graft injury; and
(h) determining that the organ is suitable for transplant when the eluate does not contain altered levels of the one or more desthiobiotinylated glycoprotein markers of the graft injury, as compared to a control eluate from a corresponding organ without the graft injury, or determining that the organ may not be suitable for transplant when the eluate contains altered levels of the one or more desthiobiotinylated glycoprotein markers of the graft injury, as compared to a control eluate from a corresponding organ without the graft injury. The organ can be a lung, a kidney, a heart, or a liver or a portion thereof. The chemical probe can include one or more of N-azidoacetylgalactosamine-tetraacylated, N-azidoacetylglucosamine-tetraacylated, and N-azidoacetylmannosamine-tetraacylated.

The mammal can be a human. The method can include perfusing the organ for about 2 to about 6 hours, or perfusing the organ for about 4 hours. The organ can be a lung, the altered levels can be elevated levels of the desthiobiotinylated glycoprotein markers, the graft injury can include warm ischemia injury, and the one or more desthiobiotinylated glycoprotein markers can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12.

In still another aspect, this document features a method for assessing an organ removed from a mammal for transplant suitability, where the method includes, or consists essentially of:
(a) perfusing the organ or the portion thereof with an aqueous solution containing an alkyne-containing chemical probe, thereby alkyne-tagging glycosylated polypeptides synthesized within cells of the organ or the portion thereof during the perfusing;
(b) obtaining a sample of the organ after the perfusing, and generating an extract containing the alkyne-tagged glycosylated polypeptides;
(c) contacting the alkyne-tagged glycosylated polypeptides in the extract with azide-desthiobiotin, thereby generating desthiobiotinylated glycosylated polypeptides;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of graft injury; and
(h) determining that the organ is suitable for transplant when the eluate does not contain altered levels of the one or more desthiobiotinylated glycoprotein markers of the graft injury, as compared to a control eluate from a corresponding organ without the graft injury, or determining that the organ may not be suitable for transplant when the eluate contains altered levels of the one or more desthiobiotinylated glycoprotein markers of the graft injury, as compared to a control eluate from a corresponding organ without the graft injury.

The organ can be a lung, a kidney, a heart, or a liver or a portion thereof. The chemical probe can include one or more of N-(4-pentynoyl)-galactosamine tetraacylated, N-(4-pentynoyl)-glucosamine tetraacylated, and N-(4-pentynoyl)-mannosamine-tetraacylated. The mammal can be a human. The method can include perfusing the organ for about 2 to about 6 hours, or perfusing the organ for about 4 hours. The organ can be a lung, the altered levels can be elevated levels of the desthiobiotinylated glycoprotein markers, the graft injury can include warm ischemia injury, and the one or more desthiobiotinylated glycoprotein markers can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12.

In another aspect, this document features a method for assessing and treating an organ removed from a mammal, where the method includes, or consists essentially of:
(a) perfusing the organ or the portion thereof with an aqueous solution containing an azide-containing chemical probe, thereby azide-tagging glycosylated polypeptides synthesized within cells of the organ or the portion thereof during the perfusing;
(b) obtaining a sample of the organ after the perfusing, and generating an extract containing the azide-tagged glycosylated polypeptides;
(c) contacting the azide-tagged glycosylated polypeptides in the extract with alkyne-desthiobiotin, thereby generating desthiobiotinylated glycosylated polypeptides;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of warm ischemia injury;
(h) determining that the eluate contains altered levels of the one or more desthiobiotinylated glycoprotein markers of graft injury, as compared to a control eluate from a corresponding organ without graft injury; and
(i) treating the organ with a calcineurin pathway inhibitor.

The organ can be a lung, a kidney, a heart, or a liver or a portion thereof. The chemical probe can include one or more of N-azidoacetylgalactosamine-tetraacylated, N-azidoacetylglucosamine-tetraacylated, and N-azidoacetylmannosamine-tetraacylated. The mammal can be a human. The method can include perfusing the organ for about 2 to about 6 hours, or perfusing the organ for about 4 hours. The organ can be a lung, the altered levels can be elevated levels, the graft injury can include warm ischemia injury, and the one or more desthiobiotinylated glycoprotein markers of warm ischemia injury can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12. The calcineurin pathway inhibitor can include cyclosporine A (CyA), tacrolimus, pimecrolimus, or voclosporin.

In yet another aspect, this document features a method for assessing and treating an organ removed from a mammal, where the method includes, or consists essentially of:
(a) perfusing the organ or the portion thereof with an aqueous solution containing an alkyne-containing chemical probe, thereby alkyne-tagging glycosylated polypeptides synthesized within cells of the organ or the portion thereof during the perfusing;
(b) obtaining a sample of the organ after the perfusing, and generating an extract containing the alkyne-tagged glycosylated polypeptides;
(c) contacting the alkyne-tagged glycosylated polypeptides in the extract with azide-desthiobiotin, thereby generating desthiobiotinylated glycosylated polypeptides;
(d) contacting the desthiobiotinylated glycosylated polypeptides with streptavidin coupled to a solid substrate, thereby coupling the desthiobiotinylated glycosylated polypeptides to the solid substrate;
(e) washing the solid substrate;
(f) eluting the desthiobiotinylated glycosylated polypeptides from the solid substrate;
(g) measuring, within the eluate, the levels of one or more desthiobiotinylated glycoprotein markers of warm ischemia injury;
(h) determining that the eluate contains altered levels of the one or more desthiobiotinylated glycoprotein markers of warm ischemia injury, as compared to a control eluate from a corresponding organ without warm ischemia injury; and
(i) treating the organ with a calcineurin pathway inhibitor.

The organ can be a lung, a kidney, a heart, or a liver or a portion thereof. The chemical probe can include one or more of N-(4-pentynoyl)-galactosamine tetraacylated, N-(4-pentynoyl)-glucosamine tetraacylated, and N-(4-pentynoyl)-mannosamine-tetraacylated. The mammal can be a human. The method can include perfusing the organ for about 2 to about 6 hours, or perfusing the organ for about 4 hours. The organ can be a lung, the altered levels can be elevated levels, the graft injury can include warm ischemia injury, and the one or more desthiobiotinylated glycoprotein markers of warm ischemia injury can include one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12. The calcineurin pathway inhibitor can be cyclosporine A (CyA), tacrolimus, pimecrolimus, or voclosporin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a schematic of a protocol for administering Ac₄GalNAz during EVLP.

FIG. 1B includes images showing histological detection of azide incorporation in tissue sections of rat lungs that received Ac₄GalNAz probe or vehicle control in the perfusate. For azide→biotin fluorescence, tissue sections were reacted to alkyne-biotin under CuAAC conditions prior to staining using fluorophore-conjugated streptavidin, to generate red staining. Laminin was stained green, and 4′,6-diamidino-2-phenylindole (DAPI) was used to stain nuclei blue. Scale bars, 50 μm. FIG. 1C includes images showing histological detection of azide incorporation in tissue sections of human lungs that received Ac₄GalNAz probe or vehicle control in the perfusate during 6 hour EVLP. For azide4biotin fluorescence, tissue sections were reacted to alkyne-biotin under CuAAC conditions prior to staining using fluorophore-conjugated streptavidin, to generate red staining. Laminin was stained green. Scale bars, 200 μm. FIG. 1D is a graph plotting azide→biotin fluorescence intensity in lung tissue sections that received Ac₄GalNAz (n=4) or vehicle (n=3), after normalization to Laminin coverage. FIG. 1E shows a western blot analysis of azide→biotin signal in extracted lung proteins using streptavidin-HRP (left) and SYPRO Ruby staining of total proteins (right). Black arrows, 75 kDa. FIG. 1F is a pair of images showing hematoxylin and eosin (H&E) staining of tissue sections of lungs that received Ac₄GalNAz or vehicle control in the EVLP perfusate. Scale bars, 1000 μm. FIG. 1G is a graph plotting averaged alveolar sizes of lungs that received Ac₄GalNAz (n=4) or vehicle (n=3), quantified with mean linear intercept of the H&E images. FIGS. 1H and 11 are graphs plotting hourly changes of lung compliance (FIG. 1H) and pulmonary vascular resistance (FIG. 1I) during EVLP for lungs that received Ac4GalNAz(n=4) or vehicle (n=5). ***, p<0.001; N.S., not statistically significant (p>0.05). Data are represented as mean±SD.

FIG. 2 includes stitched images of lung sections stained for azide-biotin and Laminin. The stained sections of lungs that received Ac₄GalNAz or vehicle control during EVLP from FIG. 1B were scanned and stitched. Azide-biotin staining was red, and Laminin staining was green. Scale bars, 2000 μm.

FIG. 3A is a schematic showing a chemoselective purification workflow for azide-tagged glycoproteins that was used in methods described herein. The tagged proteins were desthiobiotinylated (Input), pulled down by streptavidin resins (the wash-offs were saved as non-bound Supernatant), and released through competitive elution with free biotin (Eluate). FIG. 3B includes images showing western blot analysis of desthiobiotin signal in Input samples from lungs that received N-azidoacetyl-galactosamine-tetraacylated (Ac4GalNAz) or vehicle control during EVLP using streptavidin-HRP (left) and SYPRO Ruby staining of total proteins (right). FIG. 3C includes images showing western blot analysis of azide→desthiobiotin signal in Input (before resin binding) and Supernatant (after resin binding) samples from lungs that received Ac4GalNAz during EVLP. FIG. 3D includes an image showing SYPRO Ruby staining of total proteins in Eluate samples from lungs that received Ac₄GalNAz or vehicle control during EVLP. Black arrows, 70 kDa.

FIG. 4A is a schematic illustrating an affinity enrichment and proteomic analysis workflow for NewS-glycoproteins that was used in methods described herein. Protein Samples before (Inputs) or after (Eluates) affinity enrichment were analyzed by LC-MS/MS. FIGS. 4B and 4C are correlation heatmaps for proteomic profiles of Inputs (FIG. 4B) or Eluates (FIG. 4C) from lungs that received Ac₄GalNAz (n=4) or vehicle control (n=3) during EVLP. Pearson correlation analysis was performed between each pair of samples. FIG. 4D is a volcano plot for thousands of proteins identified in Ac₄GalNAz (n=4) and vehicle control (n=3) Eluates. Two-tailed t-test with Welch's correction was performed between protein intensities from Ac4GalNAz or vehicle control Eluates. X Axis, log 2 (fold change (FC): Ac4GalNAz/Vehicle). Y axis,−log 10 (p value). FC was set to the value of 1024 (Log2(FC)=10) if FC=N/A (i.e., if protein was not detected in any vehicle control Eluate sample). FC was set to the value of 0.01 (Log2(FC)=−6.64) if FC=0 (i.e., if protein was not detected in any Ac₄GalNAz Eluate samples). The proteins fell into three categories: Ac₄GalNAz-high (FC>1.5, p<0.05), non-significant between Ac₄GalNAz and vehicle control groups (p>0.05 or 2/3<FC<1.5), and vehicle-high (FC<2/3, p<0.05). FIG. 4E is a Venn diagram showing the distribution among the three categories in FIG. 4D.

FIG. 5A is a schematic illustrating the lung groups under comparison. FIG. 5B is a graph plotting lung graft function as determined by measuring the PaO₂/FiO₂ (P/F) ratio 2 hours post-LTx for the three conditions: non-LTx normal lungs in healthy rats (n=14), control-EVLP-LTx (n=5), and WII-EVLP-LTx (n=3). FIGS. 5C and 5D are graphs plotting lung compliance (FIG. 5C) and pulmonary vascular resistance (FIG. 5D) of WII and non-injured control lungs during EVLP. FIG. 5E includes images showing histological detection of azide incorporation in WII lungs that received Ac₄GalNAz during EVLP. For azide→biotin staining (red), tissue sections were reacted to alkyne-biotin under CuAAC with and without copper catalyst, followed by biotin staining using fluorophore-conjugated streptavidin. Laminin was stained green, and nuclei were stained blue with DAPI. Scale bar, 50 μm. ***, p<0.001; N.S., not statistically significant, p>0.05. Data are represented as mean±SD.

FIG. 6A is a volcano plot of each protein identified in the Eluate samples from control (n=4) or WII (n=3) lungs that received Ac4GalNAz during EVLP. Two-tailed t-test was performed between protein intensities from control and WII Eluates. X axis, log 2 (FC: WII/Control). Y axis,−log 10 (p value). The proteins fell into three categories: upregulated in WII (FC>1.5, p<0.05), non-significant (p>0.05 or 2/3<FC<1.5), and downregulated in WII (FC<2/3, p<0.05). FIG. 6B is a Venn diagram showing the distribution among the 3 categories in FIG. 6A. FIG. 6C includes graphs plotting normalized protein intensities of Itga7, Osmr, Ppp3r1, and Ca8 in WII and control lung samples both before (Input) and after (Eluate) chemoselective enrichment. FIG. 6D is a graph plotting the top enriched GO biological process terms of 120 proteins with the highest intensity rank changes between WII and control Eluates. **, p<0.01; *, p<0.05; N.S., not statistically significant, p>0.05. Data are represented as mean±SD.

FIG. 7 is a Volcano plot of the off-plot protein (Ndufaf4) in FIG. 6A. Specifically, FIG. 7 is a Volcano plot of Ndufaf4 protein intensities in Eluate samples from control (n=4) or WII (n=3) lungs that received Ac₄Ga1NAz during EVLP. Two-tailed t-test was performed between protein intensities from control and WII Eluates. X axis, log 2 (FC: WII/Control). Y axis,−log 10 (p value). Nadufaf4 was categorized as non-significant as described for FIG. 6A.

FIG. 8 includes graphs plotting normalized intensities of four proteins (Drap1, Chptl, Fads1 and Txndc15) that were down-regulated in WII versus control lung samples, showing the results both before (Inputs) and after (Eluates) chemoselective purification. **, p<0.01. *, p<0.05. N.S., not statistically significant, p>0.05. Data are represented as mean±SD.

FIGS. 9A and 9B are graphs plotting hourly lung compliance (FIG. 9A) and pulmonary vascular resistance (FIG. 9B) during EVLP of WII lungs treated with (n=4) or without (n=3) CyA. FIG. 9C includes post-LTx images of WII lungs treated with or without CyA. Naïve, naïve right lung; graft, the transplanted left lobe graft. FIG. 9D is a graph plotting post-LTx PaO₂/FiO₂ ratios for untreated (n=4) and CyA-treated (n=4) WII lungs. An outlier was identified and removed from WII-untreated group based on Grubbs's test (a=0.05). ***, p<0.001. *, p<0.01, p<0.05. N.S., not statistically significant, p>0.05. Data are represented as mean±SD.

FIG. 10 is a graph plotting wet-to-dry ratio of WII lungs treated with (n=4) or without (n=S) CyA after EVLP, before LTx. N.S., not statistically significant, p>0.05. Data are represented as mean±SD.

### DETAILED DESCRIPTION

This document provides methods and materials for the suitability of an organ or a portion thereof for transplant, based at least in part on whether one or more markers of graft injury (e.g., WII) are determined to be elevated in the organ or portion thereof. For example, this document provides methods and materials that can be used to determine whether cells of an organ (or a portion thereof) that has been removed from a mammal and has been (or is being) subjected to ex vivo perfusion contain altered (e.g., elevated or reduced) levels of one or more markers of injury (e.g., WII), as compared to reference levels of the one or more markers. The presence of an altered level of the one or more markers can indicate that the organ has a graft injury (e.g., WII) and therefore is not suitable for transplant into another mammal unless the organ is treated to alleviate or counter the effects of the injury. In some cases, the absence of an altered level of the one or more markers can indicate that the organ does not have a graft injury (e.g., WII) and therefore is suitable for transplant. This document also provides methods and materials for treating organs (e.g., organs perfused ex vivo and identified as having an elevated or reduced level of one or more markers of graft injury) ex vivo to improve their suitability for transplant. For example, an organ identified as containing cells with an elevated level of one or more markers of WII as described herein can be treated with a calcineurin pathway inhibitor to reduce the effects of the WII.

Any appropriate organ from any appropriate mammal can be assessed and, optionally, treated as described herein. Examples of mammals from which organs to be assessed and/or treated as described herein can be obtained include, without limitation, humans, non-human primates, dogs, cats, horses, cows, pigs, sheep, mice, rats, and rabbits. Examples of organs that can be assessed as described herein include, without limitation, lung, heart, kidney, and liver or portions thereof.

When assessing an organ or a portion thereof (e.g., a lung, heart, kidney, liver, or liver portion that was removed from a mammal such as a human and subjected to ex vivo perfusion) as described herein (e.g., by assessing the presence, absence, or level of one or more newly synthesized (NewS) glycoproteins in cells obtained from the organ or portion thereof), the level of any appropriate NewS glycoprotein marker of graft injury (e.g., WII) can be determined. The determined level can be compared to a reference level of the NewS glycoprotein marker to ascertain whether the determined level is elevated or reduced as compared to the reference level. For example, glycoprotein markers of WII that can be assessed as described herein include, without limitation, integrin alpha-7 (Itga7), oncostatin M receptor subunit beta (Osmr), carbonic anhydrase-related protein VIII (CA8), calcineurin subunit B type 1 (Ppp3r1), vascular cell adhesion protein 1 (Vcam1), prostaglandin G/H synthase 2 (Ptgs2), and ribosome biogenesis protein WD repeat protein 12 (Wdr12). In some cases, a method provided herein can include assessing the levels of all seven of these markers. In some cases, a method provided herein can include assessing the level of one or more of these markers (e.g., one or more, two or more, three or more, four or more, five or more, or six or more of these markers). In some cases, a method provided herein can include determining that the levels of all seven of these markers are elevated. In some cases, a method provided herein can include determining that the level of one or more of these markers (e.g., one or more, two or more, three or more, four or more, five or more, or six or more of these markers) is elevated.

As used herein, an “increased” or “elevated” level of a NewS glycoprotein in cells from an organ being evaluated (e.g., during or after ex vivo perfusion) refers to a level of the NewS glycoprotein that is higher than a reference level of the NewS glycoprotein in a control population of cells. As used herein, a “reference level” of a NewS glycoprotein can be a control level of the NewS glycoprotein in cells of a corresponding organ (e.g., an ex vivo perfused organ of the same type as the test organ) that is known to not have a graft injury (e.g., WII). In some cases, determining that an organ or portion thereof has a level of one or more NewS glycoprotein markers of graft injury (e.g., WII) that is elevated as compared to the reference level for those one or more NewS glycoprotein markers can indicate that the organ or portion thereof has a graft injury, and should likely not be transplanted without further treatment (e.g., with a calcineurin pathway inhibitor). The level of a NewS glycoprotein in cells obtained from an organ removed from a mammal and subjected to ex vivo perfusion can be considered to be “increased” or “elevated” if the level of the NewS glycoprotein is at least 5% (e.g., at least 10%, at least 15%, at least 20%, at least 25%, at least 30%, at least 40%, at least 50%, at least 75%, or more than 100%) greater than the reference level of the NewS glycoprotein in a corresponding control population of cells (e.g., cells from one or more ex vivo perfused control organs of the same type that are known to not have a graft injury such as WII).

As used herein, a “decreased” or “reduced” level of a NewS glycoprotein in cells from an organ being evaluated (e.g., during or after ex vivo perfusion) refers to a level of the NewS glycoprotein that is lower than a reference level of the NewS glycoprotein in a control population of cells. For example, the level of a NewS glycoprotein in cells obtained from an organ removed from a mammal can be considered to be “decreased” if the level of the NewS glycoprotein is at least 5% (e.g., at least 10%, at least 15%, at least 20%, at least 25%, at least 30%, at least 40%, at least 50%, at least 75%, or at least 95%) less than the level of the NewS glycoprotein in a corresponding control population of cells (e.g., cells from one or more ex vivo perfused control organs of the same type that are known to not have a graft injury such as WII).

Any appropriate method can be used to detect the presence, absence, or level of a NewS glycoprotein marker of graft injury (e.g., WII) within a cells from an organ (e.g., an organ removed from a mammal and subjected to ex vivo perfusion), to determine whether the organ is likely to have a graft injury (e.g., WII) and whether the organ is suitable for transplant into a mammal. In some cases, the presence, absence, or level of a NewS glycoprotein marker of graft injury (e.g., WII) within a sample can be determined as described in the Example herein.

In some cases, a method provided herein can include the following steps for evaluating an organ (or a portion of an organ) that is undergoing or has undergone ex vivo perfusion, to determine whether the organ or portion thereof is suitable for transplant based, at least in part, on whether the organ or portion thereof contains cells having altered (e.g., elevated or reduced) levels of one or more markers associated with graft injury.

First, the organ or portion thereof can be perfused with a solution (e.g., an aqueous solution) that contains a chemical probe that can azide-label or alkyne-label newly synthesized glycoproteins within cells of the organ or portion thereof. Any appropriate chemical probe can be used. Examples of chemical probes that can be used to azide-label newly synthesized glycoproteins include, without limitation, N-azidoacetylgalactosamine-tetraacylated, N-azidoacetylglucosamine-tetraacylated, and N-azidoacetylmannosamine-tetraacylated. Examples of chemical probes that can be used to alkyne-label newly synthesized glycoproteins include, without limitation, N-(4-pentynoyl)-galactosamine tetraacylated, N-(4-pentynoyl)-glucosamine tetraacylated, and N-(4-pentynoyl)-mannosamine-tetraacylated. Any appropriate concentration of chemical probe can be used (e.g., from about 10 μM to about 1000 μM, about 50 μM to about 500 μM, about 100 μM to about 300 μM, about 100 μM, about 150 μM, or about 200 μM). The perfusion and labeling can be carried out for any appropriate length of time (e.g., about 30 minutes to about 6 hours, about 1 hour to about 5 hours, about 2 hours to about 6 hours, about 2 hours to about 4 hours, about 2 hours, about 3 hours, or about 4 hours), and the perfusion can be carried out at any appropriate flow rate (e.g., from about 10% to about 100% of normal organ blood circulation, such as from about 400 to about 5000 mL/min for lung, or from about 90 to about 900 mL/min for kidney).

After perfusion for a suitable length of time to generate azide- or alkyne-tagged glycosylated polypeptides in the organ or portion thereof, a sample of the organ or portion thereof can be obtained for analysis of the NewS glycoproteins contained therein. A cellular extract containing the NewS glycoproteins can be prepared, and a desthiobiotin molecule can be added to the extract to generate desthiobiotinylated glycosylated polypeptides. In particular, when the NewS glycoproteins have an azide tag, alkyne-desthiobiotin can be added to the extract, and when the NewS glycoproteins have an alkyne tag, azide-desthiobiotin can be added to the extract. Any appropriate amount or concentration of desthiobiotin can be used. For example, desthiobiotin can be added to a concentration of about 1 μM to about 100 μM (e.g., about 1 to about 10 μM, about 10 to about 25 μM, about 10 to about 50 μM, about 25 to about 50 μM, about 50 to about 75 μM, about 75 to about 100 μM, about 10 μM, about 25 μM, about 50 μM, about 75 μM, or about 100 μM).

The desthiobiotinylated glycosylated polypeptides within the extract can then be enriched or purified by attaching them to a solid substrate. For example, the extract can be combined with a solid substrate having streptavidin coupled thereto, such that the desthiobiotinylated glycosylated polypeptides in the extract become attached to the solid substrate via biotin-streptavidin binding. After washing the substrate to remove non-specifically bound material, the desthiobiotinylated glycosylated polypeptides can be eluted from the substrate using any suitable method (e.g., by adding an excess of biotin to compete for binding to the streptavidin on the solid support).

The eluate can then be analyzed using any appropriate method to detect and/or measure the levels of NewS glycoproteins within the eluate. In some cases, NewS glycoproteins within the eluate can be detected and/or quantified using methods such as, without limitation, mass spectrometry (e.g., liquid chromatography/tandem mass spectrometry; LC-MS/MS), Western blotting, or enzyme-linked immunosorbent assay (ELISA). The level of particular NewS glycoproteins within the eluate can indicate whether or not the organ or portion thereof is suitable for transplant, or if additional treatment of the organ may be warranted. For example, a method provided herein can include determining that an organ or portion thereof is suitable for transplant when the eluate does not contain altered levels of a marker of graft injury (e.g., WII), as compared to a control eluate from a corresponding organ known not to have the graft injury. Conversely, a method provided herein can include determining that an organ or portion thereof may not be suitable for transplant (at least not without further treatment) when the eluate contains altered levels of a marker of graft injury (e.g., WII), as compared to a control eluate from a corresponding organ known not to have the graft injury.

In some cases, such as when it is determined that an organ or a portion thereof has, or is likely to have, a graft injury (e.g., WII) and therefore may not be suitable for transplant as is, the organ or portion thereof can be treated with an agent effective to reduce or ameliorate the effects of the graft injury. For example, an organ or portion thereof (e.g., a lung, kidney, heart, or liver or a portion thereof) can be treated with a calcineurin pathway inhibitor if it is determined that the organ or portion thereof has an elevated level of one or more makers indicative of WII.

Any appropriate calcineurin pathway inhibitor can be used to treat an organ or a portion thereof that is undergoing ex vivo perfusion prior to a potential transplant, in order to reduce the effects of a graft injury (e.g., WII) indicated by detection of an altered level of a NewS glycoprotein marker as described herein. Examples of calcineurin pathway inhibitors that can be used include, without limitation, cyclosporine A (CyA), tacrolimus, pimecrolimus, and voclosporin. A calcineurin pathway inhibitor can be administered to an organ or a portion thereof via any appropriate route, in any appropriate amount, and for any appropriate length of time. For example, a calcineurin pathway inhibitor can be added to the perfusate that is passed through the organ ex vivo. An effective amount of a calcineurin pathway inhibitor can be any amount that reduces the level or effects of graft injury (e.g., WII) in an organ or portion thereof being treated, without producing significant toxicity to the organ or portion thereof. In cases where the calcineurin pathway inhibitor CyA is used, an effective amount of the CyA can be from about 0.01 to about 10 μg/mL (e.g., from about 0.01 to about 0.1 μg/mL, from about 0.1 to about 1 μg/mL, from about 1 to about 5 μg/mL, of from about 5 to about 10 μg/mL. A calcineurin pathway inhibitor can be administered to an organ or a portion thereof for any appropriate duration. An effective duration for administering or using a calcineurin pathway inhibitor can be any duration that reduces the level or effects of graft injury (e.g., WII) in an organ or portion thereof being treated without producing significant toxicity to the organ or portion thereof. For example, the effective duration for can vary from minutes to hours (e.g., 15 to 30 minutes, 30 to 60 minutes, 1 to 2 hours, 2 to 4 hours, or 4 to 6 hours).

In some cases, the methods provided herein can be used to assess an organ that was perfused ex vivo, identified as being likely to have a graft injury (e.g., WII), and then administered a treatment as described herein (e.g., a calcineurin pathway inhibitor), to determine whether the treatment was effective. For example, an organ having been perfused with a composition containing a calcineurin pathway inhibitor can be assessed to determine whether or not the calcineurin pathway inhibitor treatment was effective, by comparing the level of the one or more markers of graft injury (e.g., WII) before and after treatment. In some cases, if the level of the one or more markers of graft injury (e.g., WII) after treatment is determined to be reduced after treatment as compared to the pre-treatment level of the one or more markers, the likelihood of graft injury (e.g., WII) can be determined to be reduced and the treatment can be deemed effective. In such cases, the organ can be transplanted into a mammalian recipient. In some cases, if the level of the one or more markers of graft injury (e.g., WII) after treatment is determined to not be reduced after treatment, as compared to the pre-treatment level of the one or more markers, the treatment can be deemed ineffective. In such cases, transplantation of the organ can be avoided.

The invention will be further described in the following example, which does not limit the scope of the invention described in the claims.

### EXAMPLE

### Example 1—Profiling of Newly-synthesized Glycoproteins to Identify Molecular Signatures of Warm Ischemic Injury in Donor Lungs

### Materials and Methods

Animals: Inbred male Lewis (RT-1I) rats weighing 250-300 g were obtained from Envigo RMS, Inc. (Indianapolis, IN). Animals were maintained in laminar flow cages in a pathogen-free animal facility and given a standard diet and water ad libitum.

Ex vivo lung perfusion (EVLP) in rats: EVLP was performed using a commercially available rodent EVLP system (IL-2 Isolated Perfused Rat or Guinea Pig Lung System; Harvard Apparatus, Holliston, MA) as described elsewhere (Noda et al., 2014, Eur J Cardio-Thorac Surg., 45:e54-60; and (Noda et al., 2021, Sci Rep., 11:12265). Acellular Steen solution (XVIVO Perfusion AB, Denver, CO) was used for perfusate and lungs were medicated with methylprednisolone (SOLU-MEDROL®; Pfizer, Inc., New York, NY) and cephalosporin (Cefazolin; WG Critical Care LLC, Paramus, NJ) equally in all experimental groups using EVLP. Perfusion flow was started at 10% of target flow and gradually increased for 1 hour toward a target flow rate that was calculated as 20% of cardiac output (75 mL/min/250 g donor body weight). Pulmonary artery pressure, peak airway pressure, and airway flow were monitored continuously, and dynamic lung compliance and pulmonary vascular resistance were also analyzed. Ac₄GalNAz (N-azidoacetylgalactosamine-tetraacylated; Click Chemistry Tools, Scottsdale, AZ, 1086) for bioorthogonal metabolic labeling was dissolved in DMSO as a stock solution and administered into the perfusate at a final concentration of 100 μM at the time of priming. To administer cyclosporine A (CyA) during EVLP, CyA (Sigma-Aldrich) was administered into the perfusate at a final concentration of 1 mM at the time of priming.

Airspace morphology evaluation: Following EVLP, the lungs receiving Ac₄GaIN Az or vehicle control administration were fixed in Formalin, embedded in paraffin, and sectioned to a thickness of 5 μm. The sections were de-paraffinized, rehydrated, and stained with hematoxylin and eosin (H&E). Three images were taken at random areas in each lung slide at a magnification of 200×. Horizontal and vertical mean linear intercepts of each image were measured by Fiji software (Schindelin et al., 2012, Exp Rev Resp Med., 11:119-128), following a protocol described elsewhere (Crowley et al., 2019, BMC Pulm Med., 19:206) The mean values of horizontal and vertical mean linear intercepts of the three images were calculated as the estimated airspace size average of each lung section.

Immunostaining: Rehydrated rat or human lung sections were incubated with antigen unmasking solution at 95° C. for 20 minutes. After cooling down, the sections were reacted with Alkyne-PEG4-biotin (Click Chemistry Tools, TA105) under copper-catalyzed cycloaddition (CuAAC) conditions (Rostovtsev et al., 2002, Angewandte Chemie., 114:2708-2711), or reacted without copper catalyst as a control. All samples were blocked with 1% bovine serum albumin (BSA) in PBS for 20 minutes and incubated with a primary antibody against Laminin (Abcam, Cambridge, UK; ab11575) at a dilution of 1:500. The slides were then stained with streptavidin conjugated to ALEXA FLUOR®-647 (Invitrogen, S21374) and Donkey-anti-Rabbit IgG conjugated to ALEXA FLUOR®-488 (Invitrogen, A32790) at a dilution of 1:500. All slides were mounted with DAPI-containing Fluoromount solution (SouthernBiotech, Birmingham, AL; 0100-20). Single-field images were taken using confocal microscopy and stitched images were taken by EVOS FL Auto 2 Imaging System (Thermo Fisher Scientific, Waltham, MA). For quantification, three random fields were taken in each lung section. Azide >biotin intensities for each field taken were measured by Fiji software (Schindelin et al., supra). Laminin channels were converted to 8-bit and intensities were measured for Laminin coverage areas. Each azide >biotin intensity was normalized by the corresponding Laminin coverage in the field. The normalized intensities from each field were averaged to represent the corresponding lung section.

Cellular protein extraction and biotinylation of rat lungs: Rat lungs after EVLP or LTx were dissected into small pieces and stored at −80° C. The frozen samples were minced into fine pieces with surgical scissors and homogenized in 2 mL tubes pre-filled with glass beads (Benchmark Scientific, Sayreville, NJ; D1031-10). The homogenized tissues were washed with PBS to remove the perfusate residues. The cellular fractions were extracted with 8 mM CHAPS buffer (with 1 M NaCl, 1% protease and phosphatase inhibitor cocktail (Thermo Fisher Scientific; 78440) in PBS, pH 5.50) at 4° C. The extracts were reacted to Alkyne-PEG4-biotin under the CuAAC condition for Western blot analysis.

Western blots and in-gel total protein staining: Protein samples were quantified using PIERCE™ BCA protein assay kit (Thermo Fisher Scientific; 23225) for equal protein input amounts, and then analyzed using SDS-PAGE. For Western blotting, the proteins in gels were transferred to nitrocellulose blotting membranes and incubated with HRP-conjugated streptavidin (Thermo Fisher Scientific; 21130) at a 1:10,000 dilution for 1 hour before autoradiography. For total protein staining, PAGE gels were fixed with 50% methanol and 7% acetic acid and stained with SYPRO Ruby protein gel stain (Thermo Fisher Scientific; S12000) overnight before visualization under UV. The blots and gels were imaged using ChemiDoc Gel and Western blot imaging system (Bio-Rad Laboratories, Hercules, CA; 12003153). Image colors of SYPRO Ruby gel stains were inverted.

Chemoselective enrichment of azide-tagged proteins: Cellular protein extracts of the lungs were reacted with Alkyne-PEG4-desthiobiotin (Click Chemistry Tools, 1109) under CuAAC conditions. Proteins were precipitated with four volumes of ice-cold acetone at −20° C. overnight to remove unreacted Alkyne-PEG4-desthiobiotin. The protein pellets were air-dried and re-dissolved in 2% SDS in PBS (pH 7.40), and then saved as Input samples. Following quantification by BCA, an aliquot of each Input sample containing 1.7 mg of protein was introduced to 75 μL streptavidin-conjugated resin (Thermo Fisher Scientific; 53117) for desthiobiotinylated protein pull-down. Following a 3-hour binding period, Supernatants were collected and the resins were washed 7 times for 15 minutes each with 2% SDS, 40 mM additional NaCl in PBS (pH 7.40). The pulled-down proteins were eluted with 30 mM biotin in 2% SDS, 40 mM additional NaCl in PBS (pH 7.40) (Eluting buffer) at room temperature for 1 hour. The eluates were concentrated with a 10 kDa molecular cut-off filter (Millipore Sigma; UFC5010) to ˜67 μL. Protein concentrations of the Eluate samples were determined by BCA assay, and the eluates were then stored at −80° C. for SDS-PAGE and proteomic analysis.

Proteomic analysis with liquid chromatography-tandem mass spectrometry (LC-MSMS): Eluate samples were further concentrated by vacuum evaporation to ˜36 μL total volume. A 4.5 μL aliquot of each Input sample was diluted to 36 μL with 4% SDS. Disulfide bonds were reduced by adding 1.5 μL of 233 mM dithiothreitol (DTT) (Sigma) and heating to 55° C. for 15 minutes after cooling. Proteins were then alkylated in the dark for 20 minutes by adding 2.3 μL of 0.5 M iodoacetamide (Sigma). Samples were acidified by adding 3.9 μL of 27.5% phosphoric acid, and subsequently diluted 6.5-fold with 240 μL of 0.1 M Tris buffer in 90% methanol. Samples were loaded into S-TrapM micro columns (Protifi, Farmingdale NY) in two aliquots by centrifuging after each addition; columns were washed four times with 150 μL of 0.1 M Tris buffer in 90% methanol. Proteolytic digestion occurred within the S-traps at 47° C. for 75 minutes after addition of 1 μg trypsin (Promega, Madison, WI) in 20 μL of 50 mM ammonium bicarbonate (ABC, Sigma). Peptides were eluted in 40 μL of 50 mM ABC, followed by 40 μL of 0.2% formic acid (Sigma), followed by 40 μL of 1:1 acetonitrile:water. Peptide samples were evaporated to dryness and then reconstituted with 10 μL of 5% acetonitrile containing 0.2% formic acid. LC-MS/MS analysis (below) was performed with 2.6 μL of each Eluate sample. A variable amount (1.0 to 2.0 μL) of each Input sample was injected to equalize their protein amounts based upon BCA results.

Samples were analyzed using a UPLC-MS/MS system consisting of an Easy-nLC 1200 ultra-high-pressure liquid chromatography system and an Orbitrap Fusion Lumos mass spectrometer (Thermo Fisher Scientific). Injected peptide samples were loaded at a pressure of 300 bar onto a 20 cm long fused silica capillary nano-column packed with C18 resin (1.7 μm-diameter, 130 A pore size from Waters). Peptides eluted over 120 minutes at a flow rate of 350 nL/min with the following gradient, where buffer A was aqueous 0.2% formic acid and buffer B was 80% acetonitrile with 0.2% formic acid: time 1 min-5% buffer B; time 52 mins-30% buffer B; time 80 mins-42% buffer B; time 90 mins-55% ACN; time 95 to 100 mins-85% buffer B; time 101 to 120 mins-equilibrate at 0% buffer B. The nano-column was held at 60° C. using a column heater constructed in-house.

The nanospray source voltage was set to 2200 V. Full-mass profile scans were performed in the orbitrap between 375-1500 m/z at a resolution of 120,000, followed by MS/MS HCD scans in the orbitrap of the highest intensity parent ions in a 3 second cycle time at 30% relative collision energy and 15,000 resolution, with a 2.5 m/z isolation window. Charge states 2-6 were included and dynamic exclusion was enabled with a repeat count of one over a duration of 30 seconds and a 10 ppm exclusion width both low and high. The AGC target was set to “standard,” maximum inject time was set to “auto,” and 1 μscan was collected for the MS/MS orbitrap HCD scans.

Proteomic data analysis: LC-MS/MS data were analyzed with the free and open-source search software program MetaMorpheus (version 0.0.320, https://github.com/smith-chem-wisc/MetaMorpheus/releases). The Swiss-Prot Rat XML (reviewed) database containing 8,137 protein entries (downloaded from UniProt 2/09/2022) was utilized, along with MetaMorpheus's contaminants database. MetaMorpheus was used to calibrate the 24 raw files and then subject them to Global Post-Translational Modification Discovery (GPTMD) (Solntsev et al., 2018, J Proteome Res., 17:1844-1851) in order to identify PTMs not annotated in the reference database including: the Common Biological, Common Artifact, and Metal Adduct categories within MetaMorpheus; as well as two custom modifications involving the azido sugar labels (on serine/threonine with and without the alkyne-biotin at +244.0808 Da and +671.3490, respectively). Next, the mass spectra files were searched against the GPTMD databases using the following search settings: protease=trypsin; maximum missed cleavages=2; minimum peptide length=7; maximum peptide length=unspecified; initiator methionine behavior=Variable; fixed modifications=Carbamidomethyl on C, Carbamidomethyl on U; variable modifications=Oxidation on M; max mods per peptide =2; max modification isoforms=1024; precursor mass tolerance=5 PPM; product mass tolerance=±20 PPM; report PSM ambiguity=True. All proteomic data, as well as the xml search database downloaded from UniProt, have been deposited in the MassIVE repository (https://massive.ucsd.edu/) under the dataset identifier MSV000090491 (also listed as ProteomeXchange dataset PXD037203).

The protein intensities were normalized as follows. Inputs: non-Rattus norvegicus proteins were removed, and then the intensity of all rat proteins were summed for each sample. The normalized intensity of protein X in sample i was calculated with the following formula:

$${X}_{i , normalized} = {X}_{j , original} * ( {Sum}_{average} / {Sum}_{i} ) ,$$

where Sum_{average} was the average sum of all the Input samples. Vehicle and Ac₄GalNAz Eluates were normalized separately due to the obvious difference in total protein intensities from the two groups. The normalized intensity of protein X in vehicle sample j was calculated with the following formula:

$${X}_{j , normalized} = {X}_{j , original} * ( {Sum}_{average} / {Sum}_{j} ) ,$$

where Sum_{average} was the average sum of all three vehicle Eluate samples. The normalized intensity of protein X in Ac₄GalNAz sample k was calculated with the following formula:

$${X}_{k , normalized} = {X}_{k , original} * ( {Sum}_{average} / {Sum}_{k} ) ,$$

where Sum_{average} was the average sum of all eight Ac₄GalNAz Eluate samples (including control and WII groups).

The Uniprot Rat Glycosylation Excel database containing 2177 protein entries (2123 reviewed and 54 unreviewed, downloaded from Uniprot 10/18/22) was utilized for glycosylation identification.

Gene ontology (GO) analysis: GO analysis was performed on DAVID Bioinformatics Resources (https://david.ncifcrf.gov/tools.jsp). The gene list was uploaded and the top GOTERM_BP_DIRECT terms with lowest p-values were enriched and plotted in bar graphs.

Establishment of WI model in rat lungs: Rats were sedated with 4% isoflurane via inhalation for tracheostomy and then placed on a ventilator, receiving 5% isoflurane with 100% O₂ via inhalation through the ventilator for 15 minutes to induce a deep state of anesthesia causing arrest of spontaneous breathing. The rats were disconnected from the ventilator (Withdrawal from Life-Sustaining Therapy, WLST). Cardiac activity and blood oxygen saturation were monitored after WLST until cardiac arrest. One hour after WLST, a median thoracotomy was performed, and blood was flushed from the lungs with low potassium dextran solution (Perfadex; XVIVO Perfusion AB) through the pulmonary artery. Then, the heart-lung bloc was isolated and stored in cold Perfadex.

Rat orthotopic left lung transplantation following EVLP: Orthotopic, single-lung transplantation of the left lung was performed using the 3-cuff method as described elsewhere (Noda et al., 2014, supra; and Noda et al., 2017, J Heart Lung Transplant., 36:466-474) After EVLP for 4 hours, the lungs were precooled with 4° C. Perfadex on the EVLP system and stored at 4° C. for 1 hour prior to LTx. Recipient animals were sacrificed 2 hours after reperfusion. At the time of analysis of graft function, the naïve lung was clamped, 100% O₂ was administered for 5 minutes through a ventilator, and the recipient's blood was sampled from the graft pulmonary vein for blood gas analysis.

Wet-to-dry ratio: The wet weight of lung tissues after EVLP was measured immediately after collection, and lung tissues were then placed into a 60° C. oven to dry for 72 hours. Tissues were weighed after drying and the wet-to-dry lung weights were determined (Haam et al., 2020, Transplant., 104:e252-e259).

Statistical analysis: For all experiments, the n values stated represent the number of independent biological samples. Data were analyzed using GraphPad software, Excel or R studio. Data with repeated measures at multiple time points (lung compliance and pulmonary vascular resistance) were analyzed by multiple t-tests with Holm-Sidak correction. Data with three or more treatment groups were analyzed by one-way ANOVA with Tukey's multiple comparisons tests. Outliners were identified and cleared out by Grubbs' tests (a=0.05). Other data were analyzed by two-tailed t-test with or without Welch's correction, as appropriate. Error bars were plotted by standard deviations. All statistical significance was reported accordingly. N.S., p>0.05. *, p<0.05. **, p<0.01. *** p<0.001.

### Results

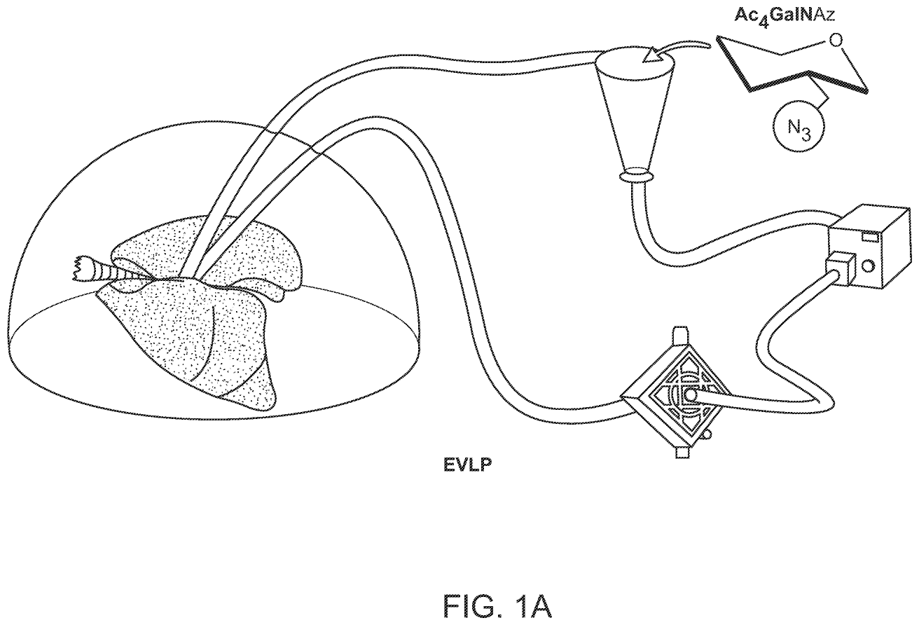

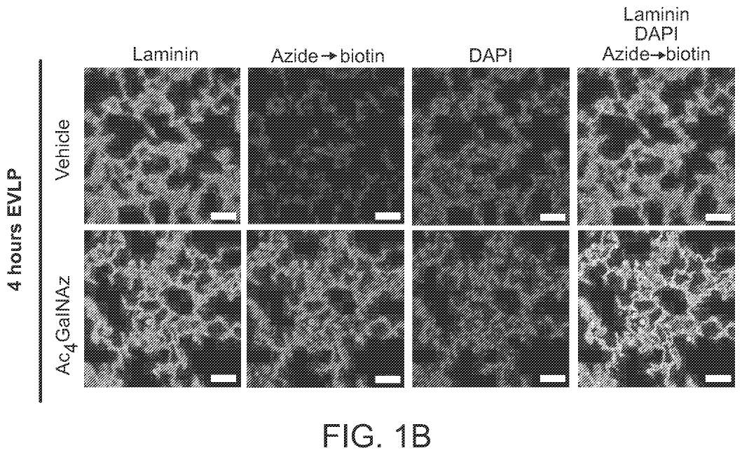

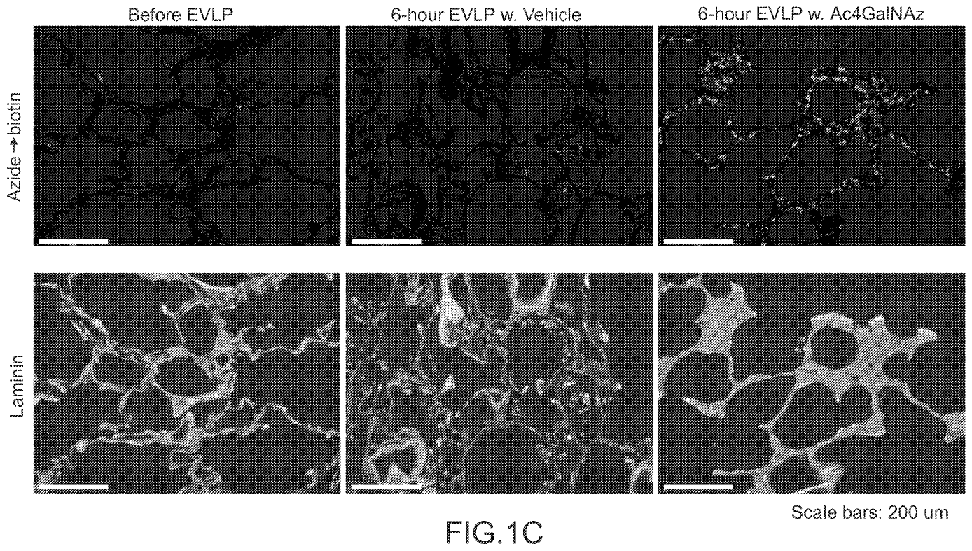

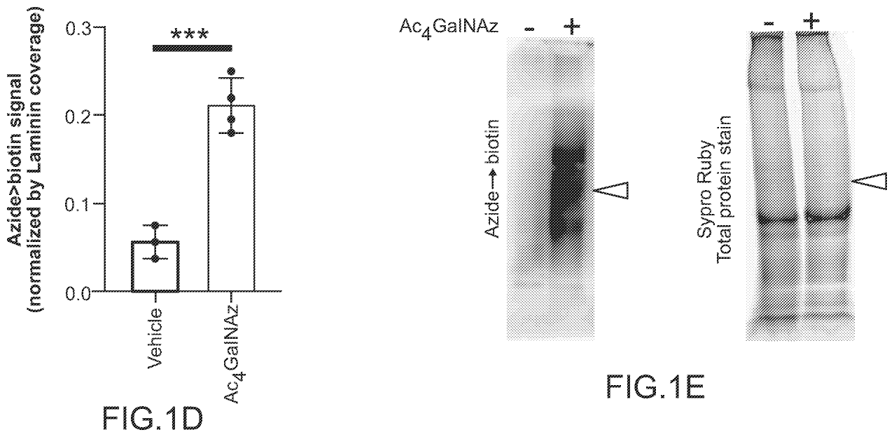

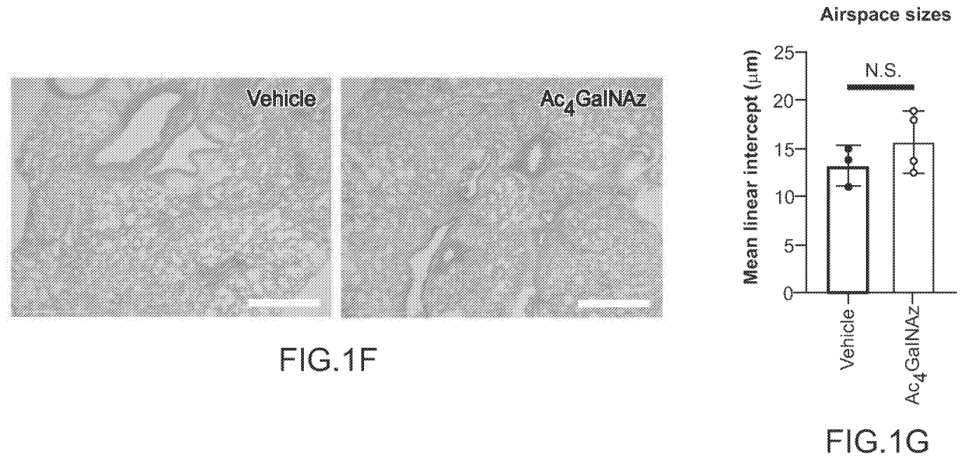

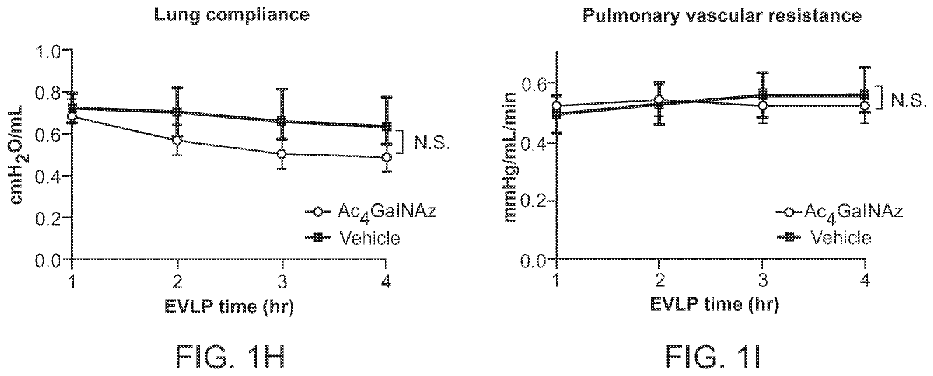

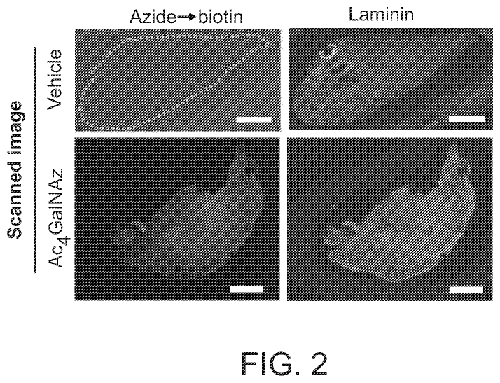

Bioorthogonal labeling of Ne'S-glycoproteins during 4-hour EVLP: A bioorthogonal approach was developed to capture and identify newly emerged protein effectors mediating the dynamic changes in lung graft quality over EVLP. This bioorthogonal approach involved selectively labeling NewS-glycoproteins that were produced during the course of EVLP through the incorporation of click chemistry-reactive, chemoselective azide tags, using healthy rat lungs as a model. To be compatible with subsequentLTx, EVLP of rat lungs typically is limited to 4-6 hours (Noda et al. 2014, supra; Noda et al. 2017, supra; and Tane et al., 2017, Chest, 151:1220-1228). To determine that metabolic labeling and analysis of NewS-glycoproteins was feasible during EVLP for4 hours, 100 μM Ac₄GalNAz or 0.1% DMSO (vehicle control) was administered in EVLP perfusate (Steen solution), and the lungs were perfused for 4 hours following organ procurement (FIG. 1A). Combining biotin-alkyne click conjugation and fluorescence staining of biotin in post-EVLP lungs, robust and specific azide labeling was observed throughout the entire lung in the Ac4GalNAz perfusate group as compared to the vehicle control (FIGS. 1B and 2). This result also was observed in human lung sections (FIG. 1C), and was confirmed by fluorescence intensity quantification of azide labeling through its biotinylation, which was normalized to the coverage of Laminin that indicated the coverage of lung parenchyma (FIG. 1D). In addition, tissue homogenization, protein extraction, and Western blot analysis were performed to validate covalent azide labeling of lung tissues at the protein level (FIG. 1E).

After establishing effective NewS-glycoprotein labeling by Ac₄GalNAz probe during EVLP, studies were conducted to assess whether the probe introduction affected lung graft quality. Histological analysis of the post-EVLP lungs revealed similar lung tissue morphology between lungs perfused with Ac4GalNAz and vehicle control (FIG. 1F). This was confirmed by the measurement of averaged alveolar sizes estimated by mean linear intercept (FIG. 1G). In addition, lung compliance and pulmonary vascular resistance were monitored every hour during the EVLP process, and no significant differences between Ac4GalNAz and vehicle perfusion groups were observed (FIGS. 1H and 1I). These results demonstrated that administering Ac4GalNAz in Steen Solution resulted in robust NewS-glycoprotein labeling during EVLP, without interfering with tissue morphology and physiology.

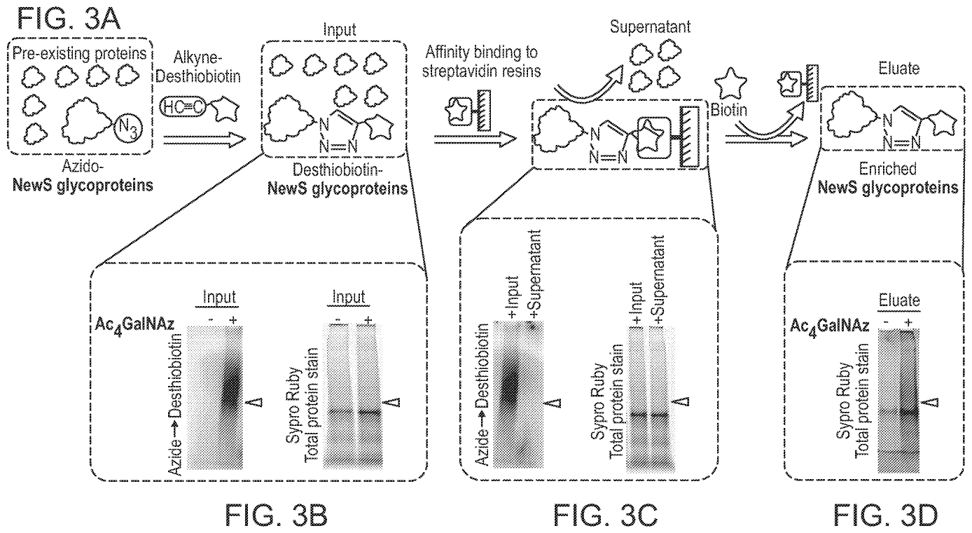

Chemoselective enrichment of NewS-glycoproteins labeled during EVLP After demonstrating the efficiency and biocompatibility of bioorthogonal glycoprotein labeling during EVLP, studies were conducted to enrich the labeled NewS-glycoproteins using alkyne-desthiobiotin click conjugation and streptavidin pull-down (FIG. 3A). To achieve this, alkyne-desthiobiotin was specifically conjugated onto azide-tagged NewS-glycoproteins from Ac₄GalNAz perfused lungs (FIG. 3B). By analyzing lung protein samples before (Input) and after (Supernatant) incubation with the streptavidin resin, the high efficiency and specificity of the binding of desthiobiotinylated NewS-glycoproteins to streptavidin resin was confirmed (FIGS. 3B and 3C). Following resin binding and extensive washing to remove unbound proteins, the bound desthiobiotinylated NewS-glycoproteins were eluted by introducing biotin, which has a higher binding affinity for streptavidin than the desthiobiotin moiety (Hirsch et al., 2002, Analyt Biochem., 308:343-357). Using SYPRO Ruby stain, proteins collected in the Eluates were visualized, revealing specific protein enrichment from the Ac4GalNAz−labeled samples (FIG. 3D). These results demonstrated effective and specific enrichment of NewS-glycoproteins bio orthogonally labeled during 4-hour EVLP.

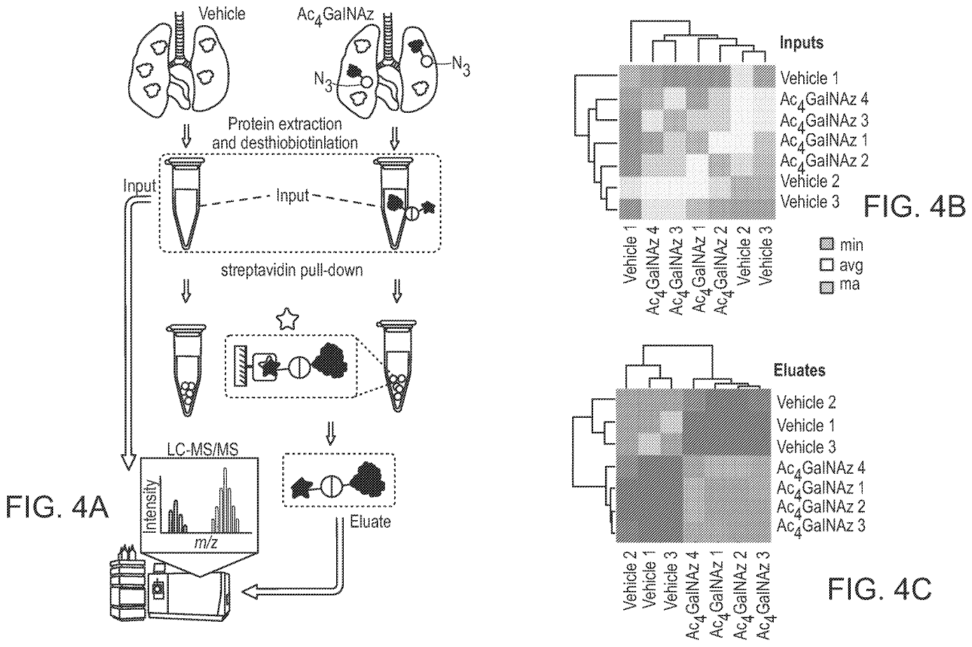

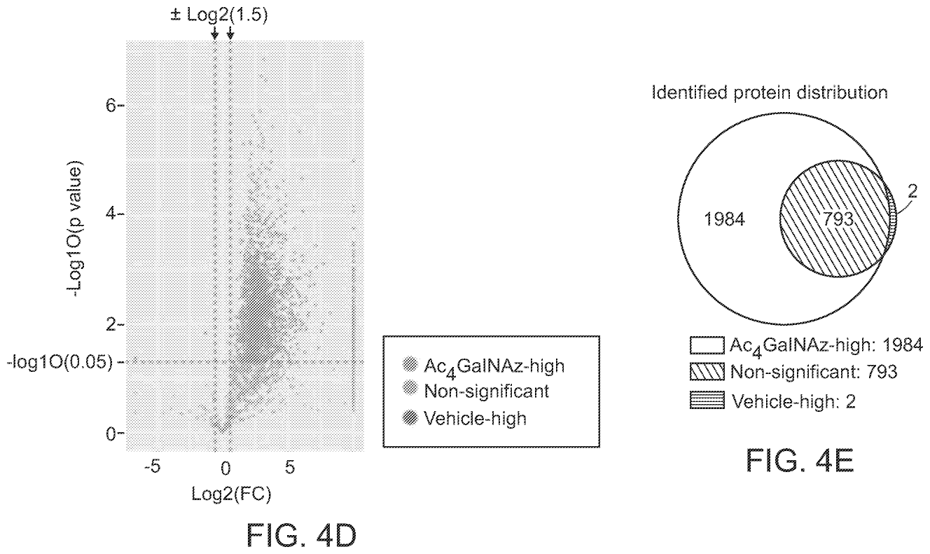

Targeted proteomic analysis of NewS-glycoproteins produced over EVLP: To determine whether Ac4GalNAz treatment affected the overall lung proteome, liquid chromatography-tandem mass spectrometry (LC-MS/MS) analysis of lung protein extracts collected prior to streptavidin affinity enrichment (referred to as “Input,” i.e., input samples for the affinity enrichment) was performed, and the proteomic profiles from Input samples derived from lung tissues with or without Ac₄GalNAz administration during EVLP were compared (FIG. 4A). After LC-MS/MS, normalized protein intensities from both groups were analyzed using Pearson correlation analysis. Hierarchical clustering showed that the Input proteomic profiles did not cluster based on the presence or absence of Ac₄TalNAz treatment (FIG. 4B), indicating that the addition of Ac₄GalNAz did not significantly alter the overall proteome of post-EVLP lungs.

LC-MS/MS analysis was then performed on the “Eluate” samples obtained after streptavidin pull-down that specifically enriched the desthiobiotinylated, azide-tagged proteins (FIG. 4A). Protein intensities from vehicle and Ac₄GalNAz samples were normalized based on their respective averaged total Eluate protein intensities. Hierarchical clustering analysis showed strong correlation among the four Eluate samples from Ac4GalNAz labeling compared to vehicle control samples, suggesting effective and consistent labelling and enrichment of NewS-glycoproteins (FIG. 4C).

The abundances of proteins detected in Eluate samples were then compared and illustrated in a volcano plot (FIG. 4D). Of the 2779 protein candidates identified in at least one Eluate sample, 1984 proteins showed significantly higher abundance in Ac₄GalNAz−lung Eluates compared to vehicle-control-lung Eluates, whereas only 2 proteins showed higher abundance in vehicle-control-lung Eluates (FIGS. 4D and 4E). These results were consistent with the findings observed with SYPRO Ruby protein staining of the Eluates (FIG. 3D), and together demonstrated effective enrichment and identification of NewS-glycoproteins that were metabolically labeled during 4-hour EVLP. Subsequent analyses were focused on the 1984 proteins that were specifically enriched in the Ac₄GalNAz−labeled lungs.

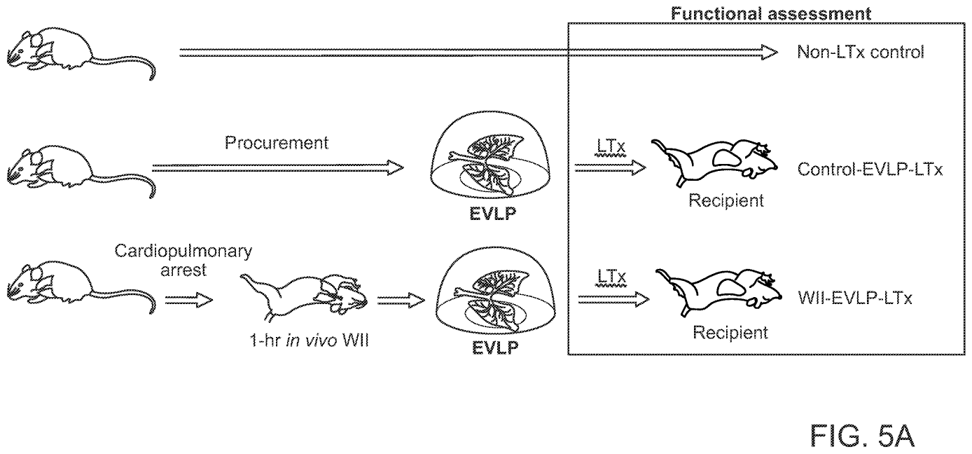

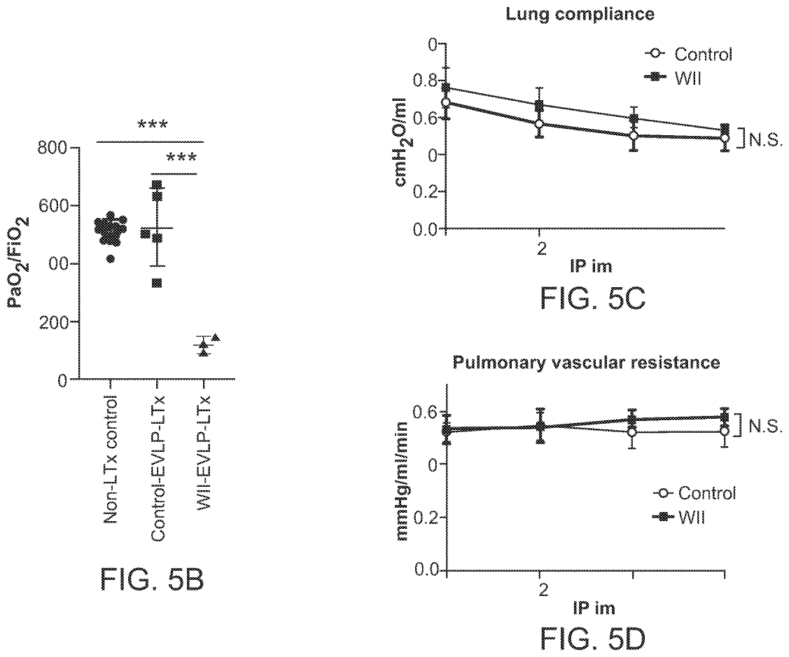

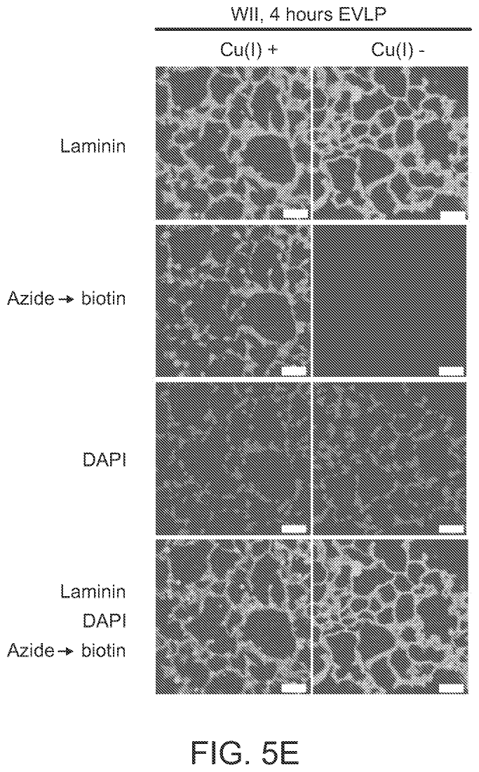

Identification of NewS-glycoprotein signatures associated with warm-ischemia injury (WI) during EVLP: The analytical pipeline was used to identify NewS-glycoprotein signatures that emerged during EVLP of lungs with WII, a common cause for primary graft dysfunction. An established rat WII-EVLP model was utilized, where a 1-hour in vivo lung WII was induced by cardiopulmonary arrest, followed by procurement and 4-hour EVLP prior to LTx (FIG. 5A). To investigate the effect of donor WII on the graft function after LTx, three groups of lungs were compared: (1) non-LTx control, corresponding to normal lungs in healthy rats; (2) control-EVLP-LTx, corresponding to transplanted lung grafts without WII but with 4-hour EVLP; and (3) WII-EVLP-LTx, corresponding to transplanted lungs with 1-hour WII and then 4-hour EVLP (FIG. 5A). While no obvious difference was identified between the non-LTx normal lungs and the control-EVLP-LTx lungs, the WII-EVLP-LTx lungs exhibited significantly lower partial pressure of oxygen/fraction of inspired oxygen (PaO₂/FiO₂)ratios compared to the other two control lung groups (FIG. 5B), suggesting an obvious defect in gas exchange efficiency of lung grafts with WII. This confirmed the validity of the WII model causing post-LTx graft dysfunction. Further, despite distinct post-LTx outcomes, WII lungs exhibited similar lung compliance (FIG. 5C), pulmonary vascular resistance (FIG. 5D), and azide labeling of NewS-glycoproteins (FIG. 5E) during EVLP compared to non-injured control lungs.

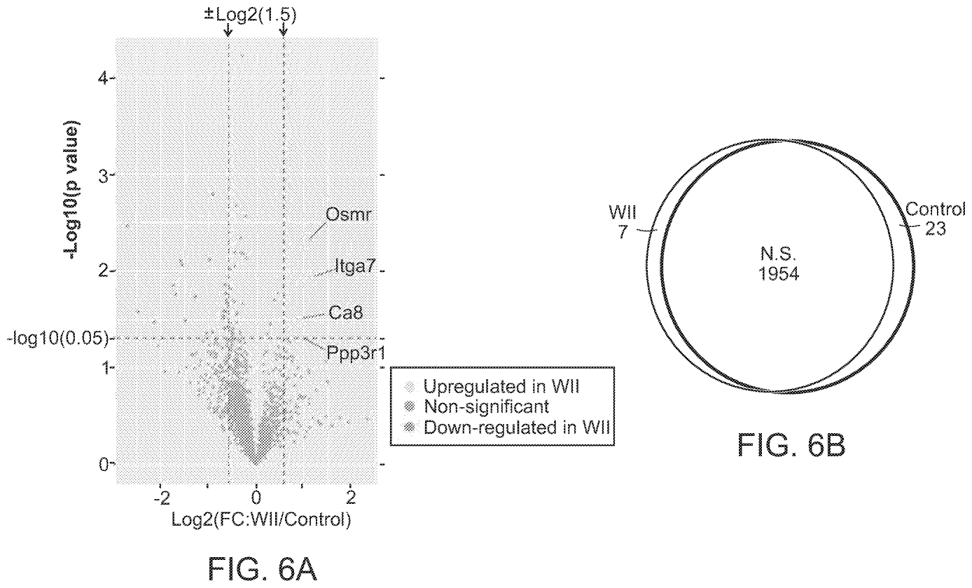

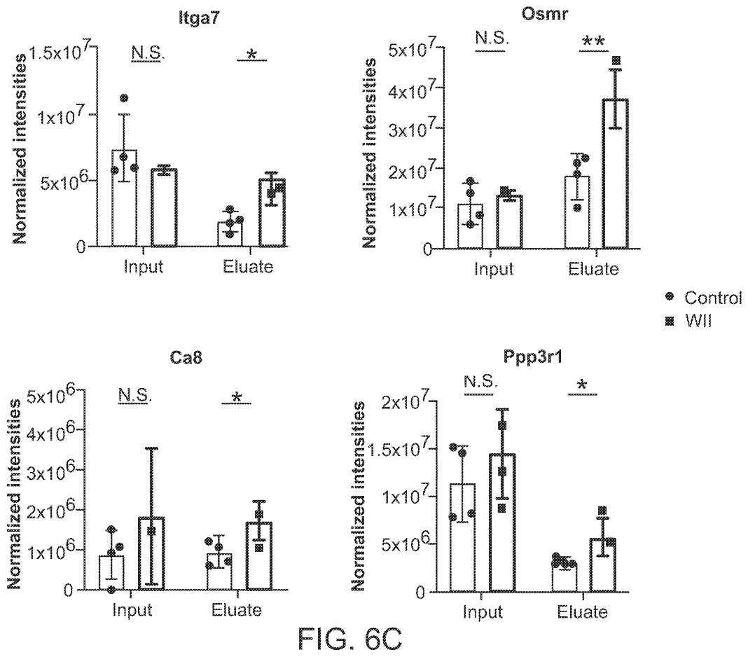

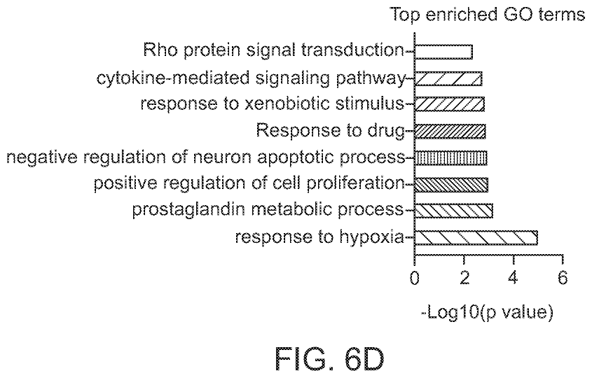

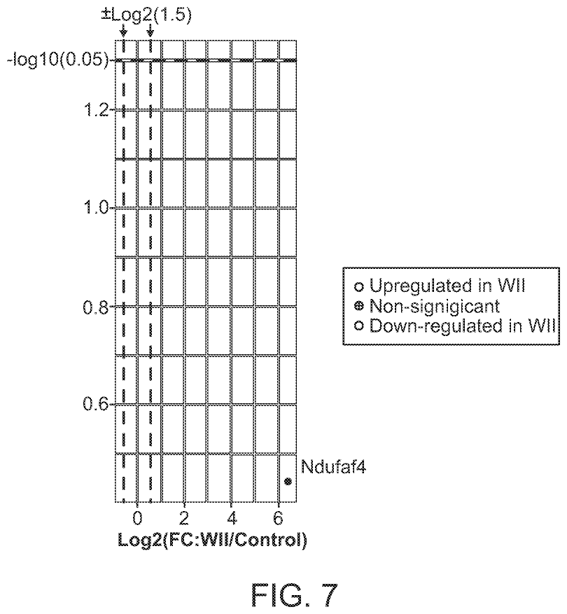

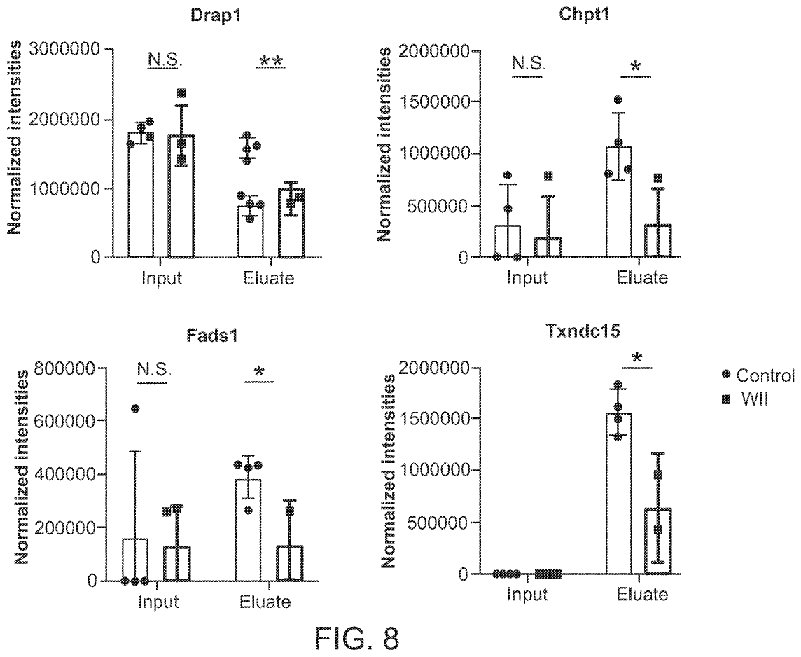

After establishing the WII-EVLP model and its associated effective bioorthogonal glycoprotein labeling, NewS-glycoprotein enrichment and comparative proteomic analysis were performed between the Eluate samples from WII and control lungs. The resulting volcano plot (FIGS. 6A and 7) and Venn diagram (FIG. 6B) showed that 7 proteins were upregulated in WII lungs, 23 proteins were downregulated in WII lungs, and 1954 proteins exhibited non-significant changes between WII and non-injured control lungs. A number of the upregulated/downregulated candidates may be involved in ischemia/reperfusion injury in a variety of organs (TABLES 1 and 2), validating the robustness of the NewS-glycoprotein profiling described in these studies. To determine whether the differences in the abundance of these identified proteins could be detected using traditional bulk proteomic analysis without NewS-glycoprotein enrichment, LC-MS/MS analysis was performed using the Input samples of WIT and control lungs. These studies showed no significant difference for most of the protein candidates (FIGS. 6C and 8, and TABLE 3). Further, due to the high background signals from the pre-existing proteins, the bulk proteomic analysis (Input) was notable to detect some low-abundance proteins, which were only identified in purified samples (Eluates) (FIG. 8, Txndc15). This demonstrated the effectiveness of the described targeted proteomic analysis based on enrichment of NewS-glycoproteins.

In addition, gene ontology (GO) analysis was conducted for the 120 proteins exhibiting the highest intensity rank changes between WII and control Eluates, as determined from their average LC-MS intensities. The top enriched GO biological process terms were response to hypoxia and prostaglandin metabolic process (FIG. 6D), and are highly connected to ischemia/reperfusion injury (Crafa et al., 1991, Eur Sur Res., 23:278-284; Frangogiannis, 2014, Nat Rev Cardiol., 11:255-265; Kuroda et al., 2012, J Hepatol., 56:146-152; Kurokawa et al., 1991, Scand J Gastroenterol. 26:269-274; Lambertsen et al., 2012, J Cereb Blood Flow Metab., 32:1677-1698; Ohata et al., 2017, Ann Thorac Surg., 103:476-483; Shimokawa et al., 2016, Circ Res., 118:352-366; and Zhang et al., 2019, Perfusion, 34:15-21), further validating the WII model and the presented NewS-glycoprotein profiling approach for identifying biologically relevant processes.

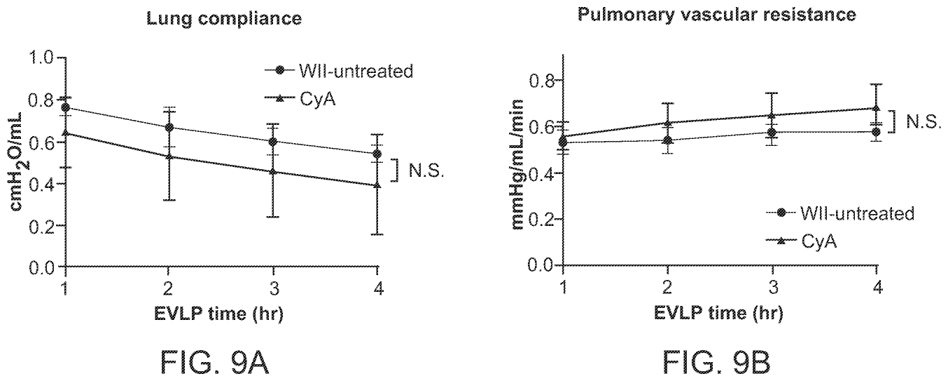

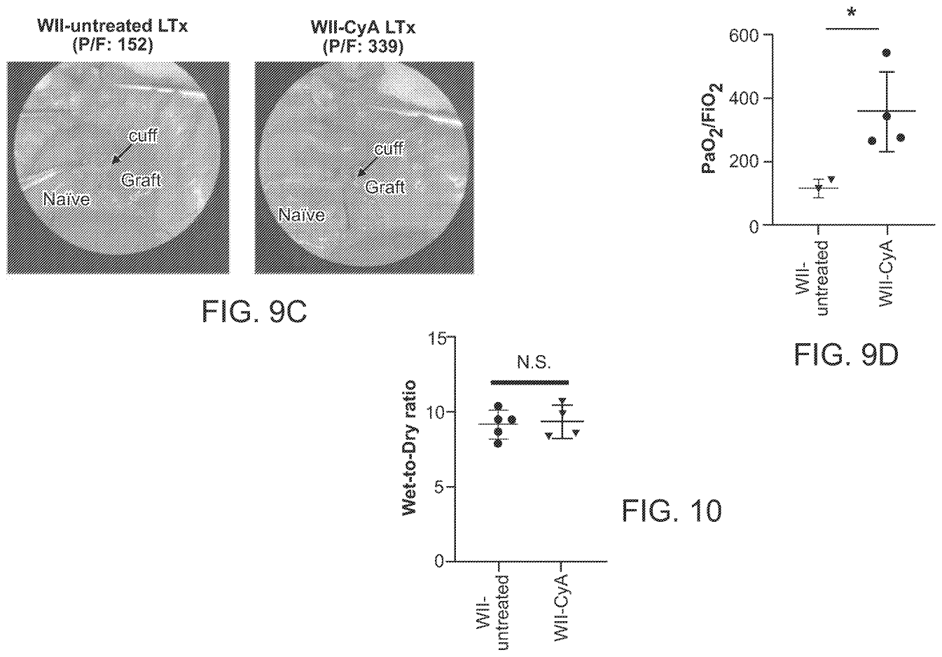

Inhibition of the calcineurin pathway during EVLP alleviates WII-induced post-transplantation graft dysfunction: Two of the seven proteins that exhibited elevated synthesis in WII lungs during EVLP, Ppp3r1 and Ca8 (TABLE 1), are associated with the calcineurin pathway that functions to dephosphorylate and activate nuclear factor of activated T cells (NFAT) and downstream expression of pro-inflammatory cytokines such as IL-2 (Matsuda and Koyasu, 2000, Ann Thorac Surg., 103:1723-1729; and Shaw et al., 1988, Science, 241:202-205). Ppp3r1 is calcineurin subunit B type 1, which regulates inflammatory response in lung grafts during EVLP (Haam et al., supra). Ca8 is involved in regulating IP3/P3R1-mediated calcium release from the intracellular membrane to the cytoplasm, and thereby plays a role in regulating the calcineurin pathway via modulating intracellular calcium concentrations (Ando et al., 2018, Proc Nat Acad Sci USA., 115:12259-12264; Dahl et al., 2000, Neurochem Int., 36:379-388; Hirota et al., 2003, Biochem J., 372:435-441; and Yuan et al., 2016, Proc Nat Acad Sci USA., 113:8532-8537). Further studies were conducted to examine the potential involvement of the calcineurin pathway in regulating WII injury in lung grafts by introducing cyclosporine A (CyA), which inhibits the phosphatase activity of calcineurin (Liu et al., 1991, Cell, 66:807-815; and Matsuda and Koyasu, supra), to the EVLP perfusate of WII lungs. Lung compliance and pulmonary vascular resistance were monitored throughout the entire 4-hour EVLP period, and graft wet-to-dry ratio was measured at the end of EVLP. These studies revealed no significant difference between WII lungs with and without CyA treatment (FIGS. 9A, 9B and 10). WII lungs were then transplanted after 4-hour EVLP and graft performance was further assessed 2 hours post-LTx. The grafts showed improved PaO₂/FiO₂ in CyA-treated WII lungs, as compared to WII lungs without CyA treatment during EVLP (FIGS. 9C and 9D). These results demonstrated that the calcineurin pathway is a positive regulator of WII in lung graft, and that inhibiting the calcineurin pathway during EVLP can partially alleviate graft damage induced by WII.

Taken together, the studies described herein established the feasibility of bioorthogonal protein labeling during EVLP, enabling chemoselective enrichment and profiling of Newly Synthesized (NewS)-glycoproteins from donor lungs. The reported analytical pipeline allowed for sensitive detection of de novo synthesized proteins in lungs during 4-hour EVLP, which are usually in low abundance, by eliminating the highly abundant pre-existing protein background. Comparing NewS-glycoprotein profiles between control and ischemic lungs, 7 upregulated NewS-glycoproteins and 23 downregulated NewS-glycoproteins were identified in WII lungs during EVLP. In addition, inhibition of the calcineurin pathway demonstrated improvement in graft recovery in WII lungs during EVLP and post-LTx function.

TABLE 1
Protein candidates with elevated synthesis in WII lungs during EVLP
and their reported connection to ischemia/reperfusion injury

| | | | Reported | |
|---|---|---|---|---|
| | | p | connection | |
| Gene | Protein | value | to I/R injury | References |
| Itga7 | Integrin alpha-7 | 0.011 | Yes | 1-2 |
| Osmr | Oncostatin M receptor subunit beta | 0.005 | Yes | 3-4 |
| CA8 | Carbonic anhydrase-related protein VIII | 0.032 | No | — |
| Ppp3r1 | Calcineurin subunit B type 1 | 0.047 | Yes | 5-7 |
| Vcam1 | Vascular cell adhesion protein 1 | 0.031 | Yes | 8-9 |
| Ptgs2 | Prostaglandin G/H synthase 2 | 0.013 | Yes | 10-12 |
| Wdr12 | Ribosome biogenesis protein WD repeat protein 12 | 0.033 | No | — |

¹Liu et al., 2020, Neural Regen Res., 15: 2123-2130
²Okada et al., 2013, J Clin Invest., 123: 4294-4308
³Brown et al., 1991, J Immunol., 147: 2175-2180
⁴Young et al., 2020, Surg Infect, 21: 799-806
⁵Bao et al., 2013, Bioactive Mat., 18: 539-551
⁶Zhang et al., 2019, J Nanobiotechnol., 17: 18
⁷Zou and Sun, 2020, J Int Med Res., 48: 300060520934627
⁸Burne et al., 2001, J Leukocyte Biol., 70: 192-198
⁹Mehaffey et al., 2017, Ann Thorac Surg., 103: 1723-1729
¹⁰Bolli et al., 2002, Cardiovasc Res., 55: 506-519
¹¹Domoki et al., 1999, Am J Physiol - Heart Circ Physiol., 277: H1207-H1214
¹²Zhu et al., 2019, Acad Radiol., 26: 404-411

TABLE 2
Protein candidates with reduced synthesis in WII lungs during EVLP

| | | | Reported | |
|---|---|---|---|---|
| | | p | connection | |
| Gene | Protein | value | to I/R injury | References |
| Drap1 | Dr1-associated corepressor | 0.003 | Yes | 1-2 |
| Pdcd4 | Programmed cell death protein 4 | 0.026 | Yes | 3-9 |
| Eef1A2 | Elongation factor 1-alpha 2 | 0.034 | Yes | 10-11 |
| Aip | aH receptor-interacting protein | 0.014 | Yes | 12-14 |
| Chpt1 | Choliephosphotransferase 1 | 0.017 | Yes | 15 |
| Gulp1 | PTB domain-containing engulfment adapter protein 1 | 0.008 | Yes | 16-17 |
| Vps52 | Vacuolar protein sorting-associated protein 52 homolog | 0.009 | No | — |
| Fads1 | Acyl-CoA (8-3)-desaturase | 0.033 | Yes | 18-23 |
| Txndc15 | Thioredoxin domain-containing protein 15 | 0.018 | No | — |
| Pdcd6 | Programmed cell death protein 6 | 0.044 | No | — |
| Dmbt1 | Deleted in malignant brain tumors 1 protein | 0.008 | Yes | 24 |
| Oas1a | 2′-5′-oligoadenylate synthase 1A | 0.043 | Yes | 25 |
| Gsta3 | Glutanione S-transferase alpha-3 | 0.002 | Yes | 26 |
| Rab12 | Ras-related protein Rab-12 | 0.030 | No | — |
| C4 | Complement C4 | 0.031 | Yes | 27 |
| Ccn2 | CCN family member 2 | 0.026 | Yes | 28-30 |
| Tcn2 | Transcobalamin-2 | 0.039 | No | — |
| Brcc3 | Lys-63-specific deubiquitinase BRCC36 | 0.014 | Yes | 31 |
| Snx1 | Sorting nexin-1 | 0.017 | No | — |
| Fmod | Fibromodulin | 0.026 | No | — |
| Apoe | Apolipoprotein E | 0.020 | Yes | 32-33 |
| Cox5b | Cytochrome c oxidase subunit 5B, mitochondrial | 0.021 | No | — |
| — | Ig kappa chain C region, B allele | 0.038 | No | — |

¹Denko et al., 2003, J Biol Chem., 278: 5744-5749
²Xu et al., 2020, J Proteom., 220: 103760
³Chen et al., 2019, Med Sci Mon., 25: 4811-4818
⁴Liu et al., 2021, Mol Med Rep., 24(1): 527
⁵Qi et al., 2021, Bioengineered 12: 5529-5539
⁶Shan et al., 2021, Cell Mol Neurobiol., 42(7): 2157-2170
⁷Xu et al., 2016, J Heart Lung Transplant., 35: S88-S89
⁸Zhang et al., 2021, Biochem Pharmacol., 186: 114466
⁹Zhu et al., 2020, Transplant Proc., 52: 383-391
¹⁰Amiri et al., 2007, Oncogene, 26: 3027-3040
¹¹Li et al., 2012, J Proteome Res., 11: 2331-2346
¹²Eleftheriadis et al., 2021, Mol Med Rep., 23(1): 41
¹³Ichihara et al., 2007, Arterioscl Thromb Vasc Biol., 27: 1297-1304
¹⁴Wang and Xu, 2019, Med Hypoth., 123: 2-5
¹⁵Feng et al., 2016, Kidney Blood Press Res., 41: 48-54
¹⁶Lee and Chung, 2021, J Pharmacol Sci., 145: 223-227
¹⁷Morizawa et al., 2017, Nat Commun., 8: 1-15
¹⁸Breil et al., 1996, Crit Care Med., 24: 1893-1902
¹⁹Koch et al., 1995, Clin Intens Care., 6: 112-120
²⁰Patchen et al., 2020, Curr Dev Nutr., 4: 1473-1473
²¹Stapleton et al., 2011, Crit Care Med., 39: 1655
²²Yang et al., 2015, Int J Clin Exp Pathol., 8: 7318-7331
²³Yin et al., 2014, J Am Coll Cardiol., 64: C117-C118
²⁴Müller et al., 2020, Pediatr Pulmonol., 55: 2964-2969
²⁵Marsh et al., 2009, Stroke, 40: S34-S37
²⁶Zhao and Yang, 2009, Chinese J Surg., 47: 373-376
²⁷Arumugam et al., 2004, Shock, 21: 401-409
²⁸Kaasbøll et al., 2016, PloS one 11: e0149000
²⁹Moe et al., 2013, J Cell Commun Sign., 7: 31-47
³⁰Valentijn et al., 2021, Antioxidants (Basel, Switzerland), 10(12): 2020
³¹Huang et al., 2021, Neurosci Lett., 756: 135954
³²Chorawala et al., 2018, Thromb Haemost., 118: 1450-1460
³³Kang et al., 2008, J Vasc Surg., 48: 701-708

TABLE 3
Normalized protein intensities between
WII and control Inputs or Eluates

| | | Statistically | | Statistically |
|---|---|---|---|---|
| | P value | significant? | P value | significant? |
| Gene name | (Eluates) | (Eluates) | (Inputs) | (Inputs) |
| Itga7 | 0.011 | Yes | 0.251 | No |
| Osmr | 0.005 | Yes | 0.458 | No |
| Ca8 | 0.032 | Yes | 0.326 | No |
| Ppp341 | 0.047 | Yes | 0.364 | No |
| Vcam1 | 0.031 | Yes | 0.345 | No |
| Ptgs2 | 0.013 | Yes | 0.002 | Yes* |
| Wdr12 | 0.033 | Yes | 0.895 | No |
| Drap1 | 0.003 | Yes | 0.858 | No |
| Pdcd4 | 0.026 | Yes | 0.469 | No |
| Eef1a2 | 0.034 | Yes | 0.168 | No |
| Aip | 0.014 | Yes | 0.249 | No |
| Chptl | 0.017 | Yes | 0.689 | No |
| Gulp1 | 0.008 | Yes | 0.421 | No |
| Vps52 | 0.009 | Yes | 0.501 | No |
| Fads1 | 0.033 | Yes | 0.874 | No |
| Txndc15 | 0.018 | Yes | N/A | No |
| Pdcd6 | 0.044 | Yes | 0.185 | No |
| Dmbt1 | 0.008 | Yes | 0.115 | No |
| Oas1a | 0.043 | Yes | 0.729 | No |
| Gsta3 | 0.002 | Yes | 0.517 | No |
| Rab12 | 0.030 | Yes | 0.758 | No |
| C4 | 0.031 | Yes | 0.343 | No |
| Ccn2 | 0.026 | Yes | 0.388 | No |
| Tcn2 | 0.039 | Yes | 0.213 | No |
| Brcc3 | 0.014 | Yes | 0.092 | No |
| Snx1 | 0.017 | Yes | 0.862 | No |
| Fmod | 0.026 | Yes | 0.414 | No |
| Apo3 | 0.020 | Yes | 0.116 | No |
| Cox5b | 0.021 | Yes | 0.413 | No |
| Ig kappa chain C | 0.038 | Yes | 0.499 | No |
| region, B allele | | | | |

*Ptgs2 was high in WII Eluates compared to Control while it was low in WII Inputs compared to Control

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for assessing an organ or a portion thereof removed from a mammal for transplant suitability, said method comprising:
(a) perfusing said organ or said portion thereof with an aqueous solution comprising (i) an azide-containing chemical probe, thereby azide-tagging glycosylated polypeptides synthesized within cells of said organ or said portion thereof during said perfusing, or (ii) an alkyne-containing chemical probe, thereby alkyne-tagging glycosylated polypeptides synthesized within cells of said organ or said portion thereof during said perfusing;
(b) obtaining a sample of said organ after said perfusing, and generating an extract containing said azide- or alkyne-tagged glycosylated polypeptides;
(c) contacting said azide- or alkyne-tagged glycosylated polypeptides in said extract with desthiobiotin, thereby generating desthiobiotinylated glycosylated polypeptides, wherein said desthiobiotin is alkyne-desthiobiotin when said glycosylated polypeptides are azide-tagged, and wherein said desthiobiotin is azide-desthiobiotin when said glycosylated polypeptides are alkyne-tagged;
(d) contacting said desthiobiotinylated glycosylated polypeptides with streptavidin coupled to a solid substrate, thereby coupling said desthiobiotinylated glycosylated polypeptides to said solid substrate;
(e) washing said solid substrate;
(f) eluting said desthiobiotinylated glycosylated polypeptides from said solid substrate;
(g) measuring, within said eluate, the levels of one or more desthiobiotinylated glycoprotein markers of graft injury; and
(h) determining that said organ is suitable for transplant when said eluate does not contain altered levels of said one or more desthiobiotinylated glycoprotein markers of said graft injury, as compared to a control eluate from a corresponding organ without said graft injury, or determining that said organ may not be suitable for transplant when said eluate contains altered levels of said one or more desthiobiotinylated glycoprotein markers of said graft injury, as compared to a control eluate from a corresponding organ without said graft injury.

2. The method of claim 1, wherein said organ is a lung, wherein said altered levels are elevated levels of said desthiobiotinylated glycoprotein markers, wherein said graft injury comprises warm ischemia injury, and wherein said one or more desthiobiotinylated glycoprotein markers comprise one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12.

3. The method of claim 1, wherein said organ is a lung, a kidney, a heart, or a liver or a portion thereof.

4. The method of claim 1, wherein said chemical probe comprises one or more of N-azidoacetylgalactosamine-tetraacylated, N-azidoacetylglucosamine-tetraacylated, and N-azidoacetylmannosamine-tetraacylated, and wherein said desthiobiotin comprises alkyne-desthiobiotin.

5. The method of claim 1, wherein said chemical probe comprises one or more of N-(4-pentynoyl)-galactosamine tetraacylated, N-(4-pentynoyl)-glucosamine tetraacylated, and N-(4-pentynoyl)-mannosamine-tetraacylated, and wherein said desthiobiotin comprises azide-desthiobiotin.

6. The method of claim 1, wherein said mammal is a human.

7. The method of claim 1, comprising perfusing said organ for about 2 to about 6 hours.

8. The method of claim 1, comprising perfusing said organ for about 4 hours.

9. A method for assessing and treating an organ removed from a mammal, said method comprising:
(a) perfusing said organ or said portion thereof with an aqueous solution comprising (i) an azide-containing chemical probe, thereby azide-tagging glycosylated polypeptides synthesized within cells of said organ or said portion thereof during said perfusing, or (ii) an alkyne-containing chemical probe, thereby alkyne-tagging glycosylated polypeptides synthesized within cells of said organ or said portion thereof during said perfusing;
(b) obtaining a sample of said organ after said perfusing, and generating an extract containing said azide- or alkyne-tagged glycosylated polypeptides;
(c) contacting said azide- or alkyne-tagged glycosylated polypeptides in said extract with desthiobiotin, thereby generating desthiobiotinylated glycosylated polypeptides, wherein said desthiobiotin is alkyne-desthiobiotin when said glycosylated polypeptides are azide-tagged, and wherein said desthiobiotin is azide-desthiobiotin when said glycosylated polypeptides are alkyne-tagged;
(d) contacting said desthiobiotinylated glycosylated polypeptides with streptavidin coupled to a solid substrate, thereby coupling said desthiobiotinylated glycosylated polypeptides to said solid substrate;
(e) washing said solid substrate;
(f) eluting said desthiobiotinylated glycosylated polypeptides from said solid substrate;
(g) measuring, within said eluate, the levels of one or more desthiobiotinylated glycoprotein markers of warm ischemia injury;
(h) determining that said eluate contains altered levels of said one or more desthiobiotinylated glycoprotein markers of graft injury, as compared to a control eluate from a corresponding organ without graft injury; and
(i) treating said organ with a calcineurin pathway inhibitor.

10. The method of claim 9, wherein said organ is a lung, wherein said altered levels are elevated levels, wherein said graft injury is warm ischemia injury, and wherein said one or more desthiobiotinylated glycoprotein markers of warm ischemia injury comprise one or more of integrin alpha-7, oncostatin M receptor subunit beta, carbonic anhydrase-related protein VIII, calcineurin subunit B type 1, vascular cell adhesion protein 1, prostaglandin G/H synthase 2, and ribosome biogenesis protein WD repeat protein 12.

11. The method of claim 9, wherein said organ is a lung, a kidney, a heart, or a liver or a portion thereof.

12. The method of claim 9, wherein said chemical probe comprises one or more of N-azidoacetylgalactosamine-tetraacylated, N-azidoacetylglucosamine-tetraacylated, and N-azidoacetylmannosamine-tetraacylated, and wherein said desthiobiotin comprises alkyne-desthiobiotin.

13. The method of claim 9, wherein said chemical probe comprises one or more of N-(4-pentynoyl)-galactosamine tetraacylated, N-(4-pentynoyl)-glucosamine tetraacylated, and N-(4-pentynoyl)-mannosamine-tetraacylated, and wherein said desthiobiotin comprises azide-desthiobiotin.

14. The method of claim 9, wherein said mammal is a human.

15. The method of claim 9, comprising perfusing said organ for about 2 to about 6 hours.

16. The method of claim 9, comprising perfusing said organ for about 4 hours.

17. The method of claim 9, wherein said calcineurin pathway inhibitor comprises one or more of cyclosporine A (CyA), tacrolimus, pimecrolimus, and voclosporin.
